SEQUENCE LISTING (41 sequences)

SEQ ID NO: 1 - DNA, 29 bp, Artificial sequence
  XK-VH1ldr
cactcgagccgccatggactggacctgga

SEQ ID NO: 2 - DNA, 18 bp, Artificial sequence
  Cmu-R4
cctgaggagacggtgacc

SEQ ID NO: 3 - DNA, 23 bp, Artificial sequence
  VK4F
gacatccagctgacccagtctcc

SEQ ID NO: 4 - DNA, 32 bp, Artificial sequence
  Kappa768 RSX
gtcgacctcgaggtcaggctggaactgaggag

SEQ ID NO: 5 - DNA, 29 bp, Artificial sequence
  XK-VH1ldr
cactcgagccgccatggactggacctgga

SEQ ID NO: 6 - DNA, 18 bp, Artificial sequence
  Cmu-R4
cctgaggagacggtgacc

SEQ ID NO: 7 - DNA, 26 bp, Artificial sequence
  VL7/8B
ccgcagdctgtggtgacycaggagcc

SEQ ID NO: 8 - DNA, 27 bp, Artificial sequence
  VL9B
ccgcagccwgkgctgactcagccmmcc

SEQ ID NO: 9 - DNA, 29 bp, Artificial sequence
  CL2R
cgccgtctagaactatgaacattctgtag

SEQ ID NO: 10 - DNA, 26 bp, Artificial sequence
  LnestR
gcactaatgcgtgacctggcagctgt

SEQ ID NO: 11 - DNA, 29 bp, Artificial sequence
  XK-VH4ldr
cactcgagccgccatggactggacctgga

SEQ ID NO: 12 - DNA, 18 bp, Artificial sequence
  Cmu-R4
cctgaggagacggtgacc

SEQ ID NO: 13 - DNA, 23 bp, Artificial sequence
  VK3F
gaaattcagctgacgcagtctcc

SEQ ID NO: 14 - DNA, 23 bp, Artificial sequence
  Kappa R3
gttattcagcaggcacacaacag

SEQ ID NO: 15 - DNA, 29 bp, Artificial sequence
  BotH1 FNEKozak
gctacgaattcgccgccatggactggacc

SEQ ID NO: 16 - DNA, 31 bp, Artificial sequence
  BotH1 H3R2
tatgaagcttgtcgaggagacggtgaccgtg

SEQ ID NO: 17 - DNA, 43 bp, Artificial sequence
  BotK sigF-C
ggatccagatctgccgccaccatggacatgagagtcctcgctc

SEQ ID NO: 18 - protein, 130 aa, Homo sapiens
QVQLVQSGAEVKKPGASVKVSCKASGYTFTSYAMHWVRQAPGQRLEWMGWINAGNGNTKYSQKFQGRVTITRDTSASTAYMELSSLRSEDTAVYYCARAALNPRGYFDWLLHYYYGMDVWGQGTTVTVSS

SEQ ID NO: 19 - DNA, 392 bp, Homo sapiens
caggtccagcttgtgcagtctggggctgaggtgaagaagcctggggcctcagtgaaggtttcctgcaaggcttctggatacaccttcactagctatgctatgcattgggtgcgccaggcccccggacaaaggcttgagtggatgggatggatcaacgctggcaatggtaacacaaaatattcacagaagttccagggcagagtcaccattaccagggacacatccgcgagcacagcctacatggagctgagcagcctgagatctgaagacacggctgtgtattactgtgcgagagcggctctaaaccctcggggatattttgactggttattacactactactacggtatggacgtctggggccaagggaccacggtcaccgtctcctcagc

SEQ ID NO: 20 - protein, 100 aa, Homo sapiens
TQSPDSLAVSLGERATINCKSSQSVLYSSNNKNYLAWYQQKPGQPPKLLIYWASTRESGVPDRFSGSGSGTDFTLTISSLQAEDVAVYYCQQYYSTPPTF

SEQ ID NO: 21 - DNA, 300 bp, Homo sapiens
acccagtctccagactccctggctgtgtctctgggcgagagggccaccatcaactgcaagtccagccagagtgttttatacagctccaacaataagaactacttagcttggtaccagcagaaaccaggacagcctcctaagctgctcatttactgggcatctacccgggaatccggggtccctgaccgattcagtggcagcgggtctgggacagatttcactctcaccatcagcagcctgcaggctgaagatgtggcagtttattactgtcagcaatattatagtactcctcccactttc

SEQ ID NO: 22 - protein, 125 aa, Homo sapiens
QVQLQESGPGLVKPSETLSLTCTVSGGSISSYYWSWIRQPPGKGLEWIGYIYYSGSTNYNPSLKSRVTISVDTSKNQFSLKLSSVTAADTAVYYCARGPTFWSGYYSVHYGMDVWGQGTTVTVSS

SEQ ID NO: 23 - DNA, 377 bp, Homo sapiens
caggtgcagctgcaggagtcgggcccaggactggtgaagccttcggagaccctgtccctcacctgcactgtctctggtggctccatcagtagttactactggagctggatccggcagcccccagggaagggactggagtggattgggtatatctattacagtgggagcaccaactacaacccctccctcaagagtcgagtcaccatatcagtagacacgtccaagaaccagttctccctgaagctgagctctgtgaccgctgcggacacggccgtgtattactgtgcgagaggcccaaccttttggagtggttattattccgtccactacggtatggacgtctggggccaagggaccacggtcaccgtctcctcagc

SEQ ID NO: 24 - protein, 95 aa, Homo sapiens
TQSPGTLSLSPGERATLSCRASQSVSSSYLAWYQQKPGQAPRLLIYGASSRATGIPDRFSGSGSGTDFTLTISRLEPEDFAVYYCQQYGSSPWTF

SEQ ID NO: 25 - DNA, 285 bp, Homo sapiens
acgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcaccgtggacgttc

SEQ ID NO: 26 - protein, 124 aa, Homo sapiens
QVQLVQSGAEVKKPGASVKVSCKASGYTFTGYYMHWVRQAPGQGLEWMGWINPNSGGTNYAQKFQGWVTMTRDTSISTAYMELSRLRSDDTAVYYCARAPLSVGFWSGYSPYYFDYWGQGTLVT

SEQ ID NO: 27 - DNA, 373 bp, Homo sapiens
caggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatcaaccctaacagtggtggcacaaactatgcacagaagtttcagggctgggtcaccatgaccagggacacgtccatcagcacagcctacatggagctgagcaggctgagatctgacgacacggccgtgtattactgtgcgagagcccccttatccgtgggtttttggagtggttattctccgtactactttgactactggggccagggaaccctggtcaccg

SEQ ID NO: 28 - protein, 96 aa, Homo sapiens
QPASVSGSPGQSITISCTGTSSDVGGYNYVSWYQQHPGKAPKLMIYDVSNRPSGVSNRFSGSKSGNTASLTISGLQAEDEADYYCSSYTSSSTWVF

SEQ ID NO: 29 - DNA, 288 bp, Homo sapiens
cagcctgcctccgtgtctgggtctcctggacagtcgatcaccatctcctgcactggaaccagcagtgacgttggtggttataactatgtctcctggtaccaacagcacccaggcaaagcccccaaactcatgatttatgatgtcagtaatcggccctcaggggtttctaatcgcttctctggctccaagtctggcaacacggcctccctgaccatctctgggctccaggctgaggacgaggctgattattactgcagctcatatacaagcagcagcacttgggtgttc

SEQ ID NO: 30 - protein, 149 aa, Homo sapiens
MDWTWRILFLVAAATGAHSQVQLVQSGAEVKKPGASVKVSCKASGYTFTSYAMHWVRQAPGQRLEWMGWINAGNGNTKYSQKFQGRVTITRDTSASTAYMELSSLRSEDTAVYYCARAALNPRGYFDWLLHYYYGMDVWGQGTTVTVSS

SEQ ID NO: 31 - DNA, 449 bp, Homo sapiens
atggactggacctggaggatcctctttttggtggcagcagccacaggtgcccactcccaggtccagcttgtgcagtctggggctgaggtgaagaagcctggggcctcagtgaaggtttcctgcaaggcttctggatacaccttcactagctatgctatgcattgggtgcgccaggcccccggacaaaggcttgagtggatgggatggatcaacgctggcaatggtaacacaaaatattcacagaagttccagggcagagtcaccattaccagggacacatccgcgagcacagcctacatggagctgagcagcctgagatctgaagacacggctgtgtattactgtgcgagagcggctctaaaccctcggggatattttgactggttattacactactactacggtatggacgtctggggccaagggaccacggtcaccgtctcctcagc

SEQ ID NO: 32 - protein, 136 aa, Homo sapiens
TQSPDSLAVSLGERATINCKSSQSVLYSSNNKNYLAWYQQKPGQPPKLLIYWASTRESGVPDRFSGSGSGTDFTLTISSLQAEDVAVYYCQQYYSTPPTFGGGTKVEIKRTVAAPSVFIFPPSDEQLKSGTASVVC

SEQ ID NO: 33 - DNA, 409 bp, Homo sapiens
acccagtctccagactccctggctgtgtctctgggcgagagggccaccatcaactgcaagtccagccagagtgttttatacagctccaacaataagaactacttagcttggtaccagcagaaaccaggacagcctcctaagctgctcatttactgggcatctacccgggaatccggggtccctgaccgattcagtggcagcgggtctgggacagatttcactctcaccatcagcagcctgcaggctgaagatgtggcagtttattactgtcagcaatattatagtactcctcccactttcggcggagggaccaaggtggagatcaaacgaactgtggctgcaccatctgtcttcatcttcccgccatctgatgagcagttgaaatctggaactgcctctgttgtgtgcc

SEQ ID NO: 34 - protein, 144 aa, Homo sapiens
MKHLWFFLLLVAAPRWVLSQVQLQESGPGLVKPSETLSLTCTVSGGSISSYYWSWIRQPPGKGLEWIGYIYYSGSTNYNPSLKSRVTISVDTSKNQFSLKLSSVTAADTAVYYCARGPTFWSGYYSVHYGMDVWGQGTTVTVSS

SEQ ID NO: 35 - DNA, 434 bp, Homo sapiens
atgaaacacctgtggttcttccttctcctggtggcagctcccagatgggtcctgtcccaggtgcagctgcaggagtcgggcccaggactggtgaagccttcggagaccctgtccctcacctgcactgtctctggtggctccatcagtagttactactggagctggatccggcagcccccagggaagggactggagtggattgggtatatctattacagtgggagcaccaactacaacccctccctcaagagtcgagtcaccatatcagtagacacgtccaagaaccagttctccctgaagctgagctctgtgaccgctgcggacacggccgtgtattactgtgcgagaggcccaaccttttggagtggttattattccgtccactacggtatggacgtctggggccaagggaccacggtcaccgtctcctcagc

SEQ ID NO: 36 - protein, 131 aa, Homo sapiens
TQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIKRTVAAPSVFIFPPSDEQLKSGTASVVC

SEQ ID NO: 37 - DNA, 395 bp, Homo sapiens
acgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcaccgtggacgttcggccaagggaccaaggtggaaatcaaacgaactgtggctgcaccatctgtcttcatcttcccgccatctgatgagcagttgaaatctggaactgcctctgttgtgtgcct

SEQ ID NO: 38 - protein, 143 aa, Homo sapiens
MDWTWRILFLVAAATGAHSQVQLVQSGAEVKKPGASVKVSCKASGYTFTGYYMHWVRQAPGQGLEWMGWINPNSGGTNYAQKFQGWVTMTRDTSISTAYMELSRLRSDDTAVYYCARAPLSVGFWSGYSPYYFDYWGQGTLVT

SEQ ID NO: 39 - DNA, 430 bp, Homo sapiens
atggactggacctggaggatcctcttcttggtggcagcagccacaggagcccactcccaggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatcaaccctaacagtggtggcacaaactatgcacagaagtttcagggctgggtcaccatgaccagggacacgtccatcagcacagcctacatggagctgagcaggctgagatctgacgacacggccgtgtattactgtgcgagagcccccttatccgtgggtttttggagtggttattctccgtactactttgactactggggccagggaaccctggtcaccg

SEQ ID NO: 40 - protein, 176 aa, Homo sapiens
QPASVSGSPGQSITISCTGTSSDVGGYNYVSWYQQHPGKAPKLMIYDVSNRPSGVSNRFSGSKSGNTASLTISGLQAEDEADYYCSSYTSSSTWVFGGGTKLTVLGQPKAAPSVTLFPPSSEELQANKATLVCLISDFYPGAVTVAWKADSSPVKAGVETTTPSKQSNNKYAASSY

SEQ ID NO: 41 - DNA, 528 bp, Homo sapiens
cagcctgcctccgtgtctgggtctcctggacagtcgatcaccatctcctgcactggaaccagcagtgacgttggtggttataactatgtctcctggtaccaacagcacccaggcaaagcccccaaactcatgatttatgatgtcagtaatcggccctcaggggtttctaatcgcttctctggctccaagtctggcaacacggcctccctgaccatctctgggctccaggctgaggacgaggctgattattactgcagctcatatacaagcagcagcacttgggtgttcggcggagggaccaagctgaccgtcctaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctac

(19) United States
(12) Patent Application Publication
(10) Pub. No.: US 2010/0222555 A1
(43) Pub. Date: Sep. 2, 2010

(54) Monoclonal antibodies that neutralize botulinum neurotoxin

(75) Inventors: Scott K. Dessain, Wynnewood, PA (US); Lance L. Simpson, Moorestown, NJ (US); Sharad P. Adekar, Secane, PA (US)

Correspondence Address: DRINKER BIDDLE & REATH;ATTN: INTELLECTUAL PROPERTY GROUP, ONE LOGAN SQUARE, SUITE 2000, PHILADELPHIA, PA, 19103-6996, US

(73) Assignee: Thomas Jefferson University, Philadelphia, PA (US)

(21) Appl. No.: 11/455,507

(22) Filed: Jun. 19, 2006

Related U.S. Application Data
(60) Provisional application No. 60/691,849, filed on Jun. 17, 2005.

(51) Int. Cl. C07K 16/12; C07K 14/00; C07H 21/04; A61K 39/395; A61P 43/00; G01N 33/53

(52) U.S. Cl. 530/387.3; 530/387.1; 530/350; 536/23.1; 424/167.1; 424/135.1; 436/501

(57) ABSTRACT

This invention provides antibodies that specifically bind to botulinum neurotoxin type A (BoNT/A) and/or botulinum neurotoxin type B (BoNT/B) and the epitopes bound by those antibodies. The antibodies and derivatives thereof and/or other antibodies that specifically bind to the neutralizing epitopes provided herein can be used to neutralize botulinum neurotoxin and are therefore also useful in the treatment of botulism. Also included in the invention are diagnostic and therapeutic assays directed to botulinum neurotoxins.

This application claims the benefit from U.S. Provisional Application No. 60/691,849, filed Jun. 17, 2005, the entire disclosure of which is incorporated herein by reference in its entirety.

### REFERENCE TO GOVERNMENT GRANT

This invention was supported in part by grant number KO8 HL04463 from the National Institutes of Health. The U.S. Government has certain rights in this invention.

### FIELD OF THE INVENTION

The invention relates to botulinum neurotoxin binding proteins, including antibodies, and DNA encoding such proteins. More specifically, the invention relates to binding proteins that specifically bind to and/or neutralize botulinum neurotoxin serotype A (BoNT/A) and/or botulinum neurotoxin serotype B (BoNT/B). The binding proteins are useful in the treatment of botulism. The invention further relates to methods of generating such proteins and DNAs.

### BACKGROUND OF THE INVENTION

Botulism is a life-threatening, flaccid paralysis caused by a neurotoxin produced by the anaerobic bacterium Clostridium botulinum. Botulinum neurotoxin poisoning (botulism) arises in a number of contexts including, but not limited to food poisoning (food borne botulism), infected wounds (wound botulism), and “infant botulism” from ingestion of spores and production of toxin in the intestine of infants. Botulism is a paralytic disease that typically begins with cranial nerve involvement and progresses caudally to involve the extremities. In acute cases, botulism can prove fatal.

Botulinum neurotoxin (BoNT) is found in nature as seven antigenically distinguishable proteins (serotypes A, B, C1, D, E, F, and G). Botulinum neurotoxin acts at neuromuscular junctions. In addition BoNT has been designated as a category A select bioterrorism agent by the United States Government because of its extreme lethality and its availability from environmental sources (Amon et al., 2001 JAMA 285:1059-70; Greenfield and Bronze, 2003 Drug Discov. Today 8:881-8; Marks, 2004 Anesthesiol. Clin. North America 22:509-32). An inhaled lethal dose of BoNT for a 70 kg person is less than 1 microgram; 1 gram contains enough BoNT to kill one million people (Amon et al., 2001 JAMA 285:1059-70). Thus, devastatingly lethal amounts of BoNT could easily be transported and distributed in secret. Because of the requirement for immediate and prolonged ICU support for exposure victims, a limited civilian exposure could easily overwhelm the intensive care unit capability of a typical American city (NIAID, 2002b).

Because C. botulinum is readily available from environmental sources, it is difficult to prevent motivated individuals from obtaining and producing BoNT. The former Soviet Union and Iraq are known to have produced BoNT for military purposes, and Iran, North Korea and Syria may have ongoing BoNT production program (Amon et al., 2001 JAMA 285:1059-70; Zilinskas, 1997 JAMA 278:418-24). Members of the cult Aum Shinrikyo exposed Japanese citizens to BoNT on at least three occasions (Amon et al., 2001 JAMA 285:1059-70). They extracted the toxin from C. botulinum obtained from a natural soil sample.

The chief countermeasures for BoNT exposure have historically been the botulinum toxoid vaccine and therapeutic antibodies. The existing vaccine is an inactivated pentavalent toxoid that induces a potent neutralizing antibody response (Amon et al., 2001 JAMA 285:1059-70; Gelzleichter et al. 1999 J. Appl. Toxicol. Suppl. 1:S35-8; Siegel, 1998 Immunol. Res. 17:239-51). However, it has not been recommended for use in the general population because the naturally occurring disease is rare and widespread vaccination would render vaccinees resistant to BoNT, which may be required for medical indications such as blepharospasm, dystonia and torticollis (Bell et al., 2000 Pharmacotherapy 20:1079-91). Use of the toxoid vaccine following BoNT exposure is of no value because it is slow to induce a neutralizing antibody response (Amon et al., 2001 JAMA 285:1059-70).

The effectiveness of therapeutic antibody treatments for BoNT exposure is well established. BoNT-neutralizing immunoglobulin (BoNT-Ig) given prior to BoNT exposure can prevent or eliminate complications (Amon et al., 2001 JAMA 285:1059-70; Gelzleichter et al. 1999 J. Appl. Toxicol. Suppl. 1:S35-8; Siegel, 1998 Immunol. Res. 17:239-51). BoNT-Ig given after exposure can prevent progression of symptoms, although it cannot reverse synaptic injury that has already occurred. However, the existing BoNT-Ig are in quantities insufficient for national defense (Amon et al., 2001 JAMA 285:1059-70). There is also a need for methods of diagnosing and treating disorders involving BoNT exposure. The present invention satisfies those needs.

### SUMMARY OF THE INVENTION

The invention provides compositions and methods for the treatment and prevention of a lethal dose of neurotoxin produced by Clostridium botulinum. As such, the present invention includes an antibody capable of specifically binding to and neutralizing BoNT/A. The invention also includes an antibody capable of specifically binding to BoNT/A and BoNT/B. The invention further includes an antibody capable of specifically binding to BoNT/B. Preferably, the antibody is produced from a hybridoma selected from the group consisting of 70A-D5, 50B-B8 and 5-3-F11-2-F8-C2. Most preferably, the antibody is selected from the antibodies designated herein 70A, 50B and 5A. The invention further provides a hybridoma selected from the group consisting of 70A-D5, 50B-B8 and 5-3-F11-2-F8-C2.

In an embodiment of the invention, the antibody specifically binds to an epitope unique to BoNT/A. Preferably, the antibody is antibody 70A. Antibody 70A comprises an antibody heavy chain variable domain having the amino acid sequence SEQ ID NO:26 and an antibody light chain having the amino acid sequence SEQ ID NO:28.

In an embodiment, the invention provides an antibody that specifically binds to an epitope specifically bound by an antibody comprising 1) an antibody heavy chain variable domain having an amino acid sequence at least 80% homology to SEQ ID NO:26; and 2) an antibody light chain variable domain having an amino acid sequence at least 80% homology to SEQ ID NO:28. In one embodiment, the antibody light chain variable domain has the amino acid sequence of SEQ ID NO:40 or at least 80% homology to SEQ ID NO:40.

In another embodiment of the invention, the antibody specifically binds to an epitope unique to BoNT/B. Preferably, the antibody is antibody 50B. Antibody 50B comprises an antibody heavy chain variable domain having the amino acid sequence SEQ ID NO:22 and an antibody light chain having the amino acid sequence SEQ ID NO:24.

In an embodiment, the invention provides an antibody that specifically binds to an epitope specifically bound by an antibody comprising: 1) an antibody heavy chain variable domain having an amino acid sequence at least 80% homology to SEQ ID NO:22; and 2) an antibody light chain variable domain having an amino acid sequence at least 80% homology to SEQ ID NO:24. In one embodiment, the antibody light chain variable domain has the amino acid sequence of SEQ ID NO:36 or at least 80% homology to SEQ ID NO:36. In one embodiment, the antibody light chain variable domain has the amino acid sequence of SEQ ID NO:36 or at least 80% homology to SEQ ID NO:36.

In yet another embodiment of the invention, the antibody specifically binds to an epitope shared by BoNT/A and BoNT/B. Preferably, the antibody is antibody 5A. More preferably, the epitope that is bound by antibody 5A is unique from the epitope bound by antibody 70A or 50B. Antibody 5A comprises an antibody heavy chain variable domain having the amino acid sequence SEQ ID NO:18 and an antibody light chain variable domain having the amino acid sequence SEQ ID NO:20.

In an embodiment, the invention provides an antibody that specifically binds to an epitope specifically bound by an antibody comprising 1) an antibody heavy chain variable domain having an amino acid sequence of SEQ ID NO: 18 or at least 80% homology to SEQ ID NO:18; and 2) an antibody light chain variable domain having an amino acid sequence of SEQ ID NO:20 or at least 80% homology to SEQ ID NO:20. In one embodiment, the antibody light chain variable domain has the amino acid sequence of SEQ ID NO:32 or at least 80% homology to SEQ ID NO:32.

The invention provides a polypeptide comprising an antibody heavy chain variable domain having the amino acid sequence selected from the group consisting of SEQ ID NO:18 or a sequence that is substantially homologous to SEQ ID NO:18. The invention also provides a polypeptide comprising an antibody light chain variable domain having the amino acid sequence SEQ ID NO:20 or a sequence that is substantially homologous to SEQ ID NO:20. The invention also provides a polypeptide comprising an antibody light chain having the amino acid sequence SEQ ID NO:32 or a sequence that is substantially homologous to SEQ ID NO:32. Corresponding nucleic acid sequences are also included in the invention.

In another aspect, the polypeptide comprises an antibody heavy chain variable domain having the amino acid sequence selected from the group consisting of SEQ ID NO:22 or a sequence that is substantially homologous to SEQ ID NO:22. In another aspect, the polypeptide comprises an antibody light chain variable domain having the amino acid sequence SEQ ID NO:24 or a sequence that is substantially homologous to SEQ ID NO:24. The invention also provides a polypeptide comprising an antibody light chain variable domain having the amino acid sequence SEQ ID NO:36 or a sequence that is substantially homologous to SEQ ID NO:36. Corresponding nucleic acid sequences are also included in the invention.

In yet another aspect, the polypeptide comprises an antibody heavy chain variable domain having the amino acid sequence selected from the group consisting of SEQ ID NO:26 or a sequence that is substantially homologous to SEQ ID NO:26. In another aspect, the polypeptide comprises an antibody light chain variable domain having the amino acid sequence SEQ ID NO:28 or a sequence that is substantially homologous to SEQ ID NO:28. The invention also provides a polypeptide comprising an antibody light chain variable domain having the amino acid sequence SEQ ID NO:40 or a sequence that is substantially homologous to SEQ ID NO:40. Corresponding nucleic acid sequences are also included in the invention.

The invention also provides a method of treating or preventing a lethal dose of botulinum neurotoxin exposure in a subject in need thereof. The method comprises administering to the subject an effective amount of an antibody that binds and neutralizes BoNT/A, wherein the antibody specifically binds to an epitope bound by an antibody comprising: 1) an antibody heavy chain variable domain having an amino acid sequence of SEQ ID NO:26 or a sequence at least 80% homology to SEQ ID NO:26; and 2) an antibody light chain variable domain having an amino acid sequence of SEQ ID NO:28 or a sequence at least 80% homology to SEQ ID NO:28. In a preferred embodiment, the antibody light chain variable domain has an amino acid sequence of SEQ ID NO:40 or a sequence at least 80% homology to SEQ ID NO:40. Preferably, the antibody is an IgM antibody. More preferably, the antibody is an IgG antibody.

A method of treating a subject exposed to BoNT/A and/or BoNT/B, or at risk for exposure to BoNT/A and/or BoNT/B, the method comprising administering to said subject an antibody of the invention. In a preferred embodiment of the invention, an antibody that binds to an epitope that is shared by BoNT/A and BoNT/B is administered.

The invention also includes a method of detecting and distinguishing between BoNT/A and BoNT/B. The method comprises binding a first antibody that specifically binds to either BoNT/A or BoNT/B to a solid phase surface; applying a test sample over said first antibody; washing away unbound material from said first antibody; and applying a second antibody that specifically binds an epitope that is shared by BoNT/A and BoNT/B. Preferably, the antibodies of the present invention are used in a sandwich assay to detect and distinguish between BoNT/A and BoNT/B.

### ABBREVIATIONS AND SHORT FORMS

The following abbreviations and short forms are used in this specification.

“BoNT/A” means botulinum neurotoxin serotype A.

“BoNT/B” means botulinum neurotoxin serotype B.

“ELISA” means enzyme-linked immunosorbent assay.

“huMAb” means human monoclonal antibody.

“Ig H” means immunoglobulin heavy chain.

“Ig L” means immunoglobulin light chain.

“MAb” means monoclonal antibody.

“PCR” means polymerase chain reaction.

“rhuMAb” means recombinant human monoclonal antibody.

“RT-PCR” means reverse transcription PCR.

“scFv” means single chain variable fragment.

“VNA” means virus-neutralization antibody.

### DEFINITIONS

The definitions used in this application are for illustrative purposes and do not limit the scope of the invention.

The articles “a” and “an” are used herein to refer to one or to more than one (e.g., to at least one) of the grammatical object of the article. By way of example, “an element” means one element or more than one element.

As used herein, each “amino acid” is represented by the full name thereof, by the three letter code corresponding thereto, or by the one-letter code corresponding thereto, as indicated in the following table:

| | Three-Letter | One-Letter |
|---|---|---|
| Full Name | Code | Code |
| Aspartic Acid | Asp | D |
| Glutamic Acid | Glu | E |
| Lysine | Lys | K |
| Arginine | Arg | R |
| Histidine | His | H |
| Tyrosine | Tyr | Y |
| Cysteine | Cys | C |
| Asparagine | Asn | N |
| Glutamine | Gln | Q |
| Serine | Ser | S |
| Threonine | Thr | T |
| Glycine | Gly | G |
| Alanine | Ala | A |
| Valine | Val | V |
| Leucine | Leu | L |
| Isoleucine | Ile | I |
| Methionine | Met | M |
| Proline | Pro | P |
| Phenylalanine | Phe | F |
| Tryptophan | Trp | W |

The expression “amino acid” as used herein is meant to include both natural and synthetic amino acids, and both D and L amino acids. “Standard amino acid” means any of the twenty L-amino acids commonly found in naturally occurring peptides. “Nonstandard amino acid residues” means any amino acid, other than the standard amino acids, regardless of whether it is prepared synthetically or derived from a natural source. As used herein, “synthetic amino acid” also encompasses chemically modified amino acids, including but not limited to salts, amino acid derivatives (such as amides), and substitutions. Amino acids contained within the peptides of the present invention, and particularly at the carboxy- or amino-terminus, can be modified by methylation, amidation, acetylation or substitution with other chemical groups which can change a peptide's circulating half life without adversely affecting activity of the peptide. Additionally, a disulfide linkage may be present or absent in the peptides of the invention.

The term “amino acid” is used interchangeably with “amino acid residue,” and may refer to a free amino acid and to an amino acid residue of a peptide. It will be apparent from the context in which the term is used whether it refers to a free amino acid or a residue of a peptide.

Amino acids have the following general structure:

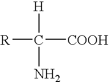

Amino acids are classified into seven groups on the basis of the side chain R: (1) aliphatic side chains, (2) side chains containing a hydroxylic (OH) group, (3) side chains containing sulfur atoms, (4) side chains containing an acidic or amide group, (5) side chains containing a basic group, (6) side chains containing an aromatic ring, and (7) proline, an amino acid in which the side chain is fused to the amino group.

The nomenclature used to describe the peptide compounds of the present invention follows the conventional practice wherein the amino group is presented to the left and the carboxy group to the right of each amino acid residue. In the formulae representing selected specific embodiments of the present invention, the amino- and carboxy-terminal groups, although not specifically shown, will be understood to be in the form they would assume at physiologic pH values, unless otherwise specified.

As used herein, “antibody” refers to intact molecules, as well as to fragments thereof, such as Fab, F(ab′)₂, Fv fragments, and single chain variable fragments (scFv), which are capable of binding an epitopic determinant. Antibody fragments refer to antigen-binding immunoglobulin peptides which are at least about 5 to about 15 amino acids or more in length, and which retain some biological activity or immunological activity of an immunoglobulin. Antibody as used herein includes polyclonal and monoclonal antibodies, hybrid, single chain, and humanized antibodies, as well as Fab fragments, including the products of an Fab or other immunoglobulin expression library.

An “antibody heavy chain,” as used herein, refers to the larger of the two types of polypeptide chains present in all antibody molecules in their naturally occurring conformations.

An “antibody light chain,” as used herein, refers to the smaller of the two types of polypeptide chains present in all antibody molecules in their naturally occurring conformations.

As used herein, the term “monoclonal antibody” includes antibodies which display a single binding specificity and affinity for a particular epitope. These antibodies are mammalian-derived antibodies, including murine, human and humanized antibodies. The term “human monoclonal antibody” as used herein, refers to antibodies displaying a single binding specificity which have variable and constant regions derived from human germ-line immunoglobulin sequences.

“Biologically active,” as used herein with respect to botulinum neurotoxin neutralizing antibodies, fragments, derivatives, homologs, and analogs means that the antibodies, fragments, derivatives, homologs or analogs have the ability to neutralize a botulinum neurotoxin, as described herein (e.g. BoNT/A and/or BoNT/B).

“Derivative” includes any purposefully generated peptide which in its entirety, or in part, comprises a amino acid sequence substantially similar to a variable domain amino acid sequence of an antibody selected from the group consisting of 70A, 50B and 5A. Antibody 70A comprises an antibody heavy chain variable domain having the amino acid sequence SEQ ID NO:26 and an antibody light chain having the amino acid sequence SEQ ID NO:28. Antibody 50B comprises an antibody heavy chain variable domain having the amino acid sequence SEQ ID NO:22 and an antibody light chain having the amino acid sequence SEQ ID NO:24. Antibody 5A comprises an antibody heavy chain variable domain having the amino acid sequence SEQ ID NO:18 and an antibody light chain having the amino acid sequence SEQ ID NO:20. Derivatives of the antibodies of the present invention may be characterized by single or multiple amino acid substitutions, deletions, additions, or replacements. These derivatives may include (a) derivatives in which one or more amino acid residues are substituted with conservative or non-conservative amino acids; (b) derivatives in which one or more amino acids are added; (c) derivatives in which one or more of the amino acids of the amino acid sequence of the invention includes a substituent group; (d) derivatives in which amino acid sequences of the invention or a portion thereof is fused to another peptide (e.g., serum albumin or protein transduction domain); (e) derivatives in which one or more nonstandard amino acid residues (e.g., those other than the 20 standard L-amino acids found in naturally occurring proteins) are incorporated or substituted into the amino acid sequences of the invention; (f) derivatives in which one or more non-amino acid linking groups are incorporated into or replace a portion of the amino acids of the invention; and (g) derivatives in which one or more amino acid is modified by glycosylation.

As used herein, an “effective amount” or “therapeutically effective amount” of botulinum neurotoxin neutralizing antibodies, is an amount sufficient to neutralize (mitigate or eliminate) BoNT/A and/or BoNT/B toxin (e.g., reduce or eliminate a symptom of BoNT/A and/or BoNT/B poisoning (botulism)).

The term “expression,” as used with respect to a botulinum neurotoxin neutralizing antibody mRNA, refers to transcription of a botulinum neurotoxin neutralizing heavy or light chain nucleic acid sequence, resulting in synthesis of botulinum neurotoxin neutralizing antibody mRNA. “Expression,” as used with respect to a botulinum neurotoxin neutralizing antibody, refers to translation of a botulinum neurotoxin neutralizing antibody mRNA, resulting in synthesis of a botulinum neurotoxin neutralizing antibody.

As used herein, the term “fragment,” as applied to a nucleic acid, refers to a subsequence of a larger nucleic acid. A “fragment” of a nucleic acid can be at least about 20 nucleotides in length; for example, at least about 50 nucleotides to about 100 nucleotides; preferably at least about 100 to about 500 nucleotides, more preferably at least about 500 to about 1000 nucleotides, even more preferably at least about 1000 nucleotides to about 1500 nucleotides; particularly, preferably at least about 1500 nucleotides to about 2500 nucleotides; most preferably at least about 2500 nucleotides.

As used herein, the term “fragment,” as applied to a protein or peptide, refers to a subsequence of a larger protein or peptide. A “fragment” of a protein or peptide can be at least about 20 amino acids in length; for example at least about 50 amino acids in length; more preferably at least about 100 amino acids in length, even more preferably at least about 200 amino acids in length, particularly preferably at least about 300 amino acids in length, and most preferably at least about 400 amino acids in length.

As used herein, the term “gene” refers to an element or combination of elements that are capable of being expressed in a cell, either alone or in combination with other elements. In general, a gene comprises (from the 5′ to the 3′ end): (1) a promoter region, which includes a 5′ nontranslated leader sequence capable of functioning in any cell such as a prokaryotic cell, a virus, or a eukaryotic cell (including transgenic animals); (2) a structural gene or polynucleotide sequence, which codes for the desired protein; and (3) a 3′ nontranslated region, which typically causes the termination of transcription and the polyadenylation of the 3′ region of the RNA sequence. Each of these elements is operably linked by sequential attachment to the adjacent element. A gene comprising the above elements is inserted by standard recombinant DNA methods into a plant expression vector.

As used herein, “gene products” include any product that is produced in the course of the transcription, reverse-transcription, polymerization, translation, post-translation and/or expression of a gene. Gene products include, but are not limited to, proteins, polypeptides, peptides, peptide fragments, or polynucleotide molecules.

“Homologous” as used herein, refers to the subunit sequence similarity between two polymeric molecules, e.g., between two nucleic acid molecules, such as, two DNA molecules or two RNA molecules, or between two polypeptide molecules. When a subunit position in both of the two molecules is occupied by the same monomeric subunit; e.g., if a position in each of two DNA molecules is occupied by adenine, then they are homologous at that position. The homology between two sequences is a direct function of the number of matching or homologous positions; e.g., if half (e.g., five positions in a polymer ten subunits in length) of the positions in two sequences are homologous, the two sequences are 50% homologous; if 90% of the positions (e.g., 9 of 10), are matched or homologous, the two sequences are 90% homologous. By way of example, the DNA sequences 3′ATTGCC5′ and 3′TATGGC5′ are 50% homologous.

As used herein, “homology” is used synonymously with “identity.”

The term “hybridoma,” as used herein refers to a cell resulting from the fusion of a spleen cell and a myeloma cell. A hybridoma can be cloned and maintained indefinitely in cell culture and is able to produce monoclonal antibodies. A hybridoma can also be considered to be a hybrid cell.

The term “inhibit,” as used herein, means to suppress or block an activity or function by at least about ten percent relative to a control value. Preferably, the activity is suppressed or blocked by 50% compared to a control value, more preferably by 75%, and even more preferably by 95%.

“Isolated” means altered or removed from the natural state through the actions of a human being. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

A “mutation,” as used herein, refers to a change in nucleic acid or polypeptide sequence relative to a reference sequence (which is preferably a naturally-occurring normal or “wild-type” sequence), and includes translocations, deletions, insertions, and substitutions/point mutations. A “mutant,” as used herein, refers to either a nucleic acid or protein comprising a mutation.

“Neutralize,” as used herein, means to inhibit the biological activity of a botulinum neurotoxin. Preferably, “neutralize,” as used herein with respect to a botulinum neurotoxin, means to reduce or inhibit progression of a botulinum neurotoxin exposure in a subject or to reduce or prevent progression in a subject at risk of exposure to a botulinum neurotoxin. Preferred antibodies of this invention act to neutralize (reduce or eliminate) the toxicity of botulinum neurotoxin.

A “nucleic acid” refers to a polynucleotide and includes poly-ribonucleotides and poly-deoxyribonucleotides.

The term “oligonucleotide” typically refers to short polynucleotides of about 50 nucleotides or less in length. It will be understood that when a nucleotide sequence is represented herein by a DNA sequence (e.g., A, T, G, and C), this also includes the corresponding RNA sequence (e.g., a, u, g, c) in which “u” replaces “T”.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids which can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptide, homodimers, heterodimers, variants of polypeptides, modified polypeptides, derivatives, analogs, fusion proteins, among others. The polypeptides include natural peptides, recombinant peptides, synthetic peptides, or a combination thereof.

As used herein, “polynucleotide” includes cDNA, RNA, DNA/RNA hybrid, anti-sense RNA, ribozyme, genomic DNA, synthetic forms, and mixed polymers, both sense and antisense strands, and may be chemically or biochemically modified to contain non-natural or derivatized, synthetic, or semi-synthetic nucleotide bases. Also, included within the scope of the invention are alterations of a wild type or synthetic gene, including but not limited to deletion, insertion, substitution of one or more nucleotides, or fusion to other polynucleotide sequences, provided that such changes in the primary sequence of the gene do not alter the expressed peptide ability to elicit passive immunity.

“Pharmaceutically acceptable” means physiologically tolerable, for either human or veterinary applications.

As used herein, “pharmaceutical compositions” include formulations for human and veterinary use.

A “preventive” or “prophylactic” treatment is a treatment administered to a subject who does not exhibit signs, or exhibits only early signs, of botulinum neurotoxin exposure or infection of C. botulinum. A prophylactic or preventative treatment is administered for the purpose of decreasing the risk of developing pathology associated with botulinum neurotoxin exposure or infection of C. botulinum.

“Botulinum neurotoxin or C. botulinum-associated disorder,” as used herein, refers to a disorder in which there is an association between the presence of botulinum neurotoxin exposure or infection of C. botulinum and clinical signs thereof.

“Botulinum neurotoxin-neutralizing,” as used herein with respect to recombinant human antibodies, refers to an antibody or mixture of antibodies which exhibits the ability to reduce the extent to which a botulinum neurotoxin exposure or infection of C. botulinum elicits a disease/disorder state in an animal. “Botulinum neurotoxin-neutralizing” is used interchangeably with “C. botulinum-neutralizing activity.”

As used herein, “promoter” refers to a region of a DNA sequence active in the initiation and regulation of the expression of a structural gene. This sequence of DNA, usually upstream to the coding sequence of a structural gene, controls the expression of the coding region by providing the recognition for RNA polymerase and/or other elements required for transcription to start at the correct site.

As used herein, “protecting group,” with respect to a terminal amino group of a peptide, refers to a terminal amino group of the peptide which is coupled to any of various amino-terminal protecting groups traditionally employed in peptide synthesis. Such protecting groups include, for example, acyl protecting groups such as formyl, acetyl, benzoyl, trifluoroacetyl, succinyl, and methoxysuccinyl; aromatic urethane protecting groups such as benzyloxycarbonyl; and aliphatic urethane protecting groups, for example, tert-butoxycarbonyl or adamantyloxycarbonyl. See Gross and Mienhofer, eds., The Peptides, vol. 3, pp. 3-88 (Academic Press, New York, 1981) for suitable protecting groups.

As used herein, “RT-PCR” refers to reverse transcription-polymerase chain reaction, which is a technique whereby an RNA strand is “reverse” transcribed into its DNA complement, followed by amplification of the resulting DNA using a polymerase chain reaction (PCR).

A “sample,” as used herein, refers to a biological sample from a subject, including normal tissue samples, blood, saliva, feces, or urine. A sample can also be any other source of material obtained from a subject which contains a compound or cells of interest.

As used herein, an antibody “specifically binds,” referring to an antibody binding to Botulinum neurotoxin, means that the antibody binds a Botulinum neurotoxin polypeptide, or fragment thereof, but does not bind to a non-Botulinum neurotoxin polypeptide. Antibodies that specifically bind to a Botulinum neurotoxin, or fragment thereof, do not cross-react with antigens outside of the family of Botulinum neurotoxins. A “subject,” as used herein, can be a human or non-human animal. Non-human animals include, for example, livestock and pets, such as ovine, bovine, porcine, canine, feline and murine mammals, as well as reptiles, birds and fish. Preferably, the subject is a human.

As disclosed herein, “substantially homologous sequences” include those sequences which have at least about 50% homology, preferably at least about 60%, more preferably at least about 70% homology, even more preferably at least about 80% homology, more preferably 85% homology, yet more preferably 90% homology, more preferably at least about 95%, and most preferably at least about 99% or more homology to the sequences of the invention.

As used herein, a “substantially homologous amino acid sequences” includes those amino acid sequences which have at least about 50% homology, preferably at least about 60%, more preferably at least about 70% homology, even more preferably at least about 80% homology, more preferably 85% homology, yet more preferably 90% homology, even more preferably at least about 95%, and most preferably at least about 99% or more homology to an amino acid sequence of a reference antibody chain. Amino acid sequence similarity or identity can be computed by using the BLASTP and TBLASTN programs which employ the BLAST (basic local alignment search tool) 2.0.14 algorithm. The default settings used for these programs are suitable for identifying substantially similar amino acid sequences for purposes of the present invention.

“Substantially homologous nucleic acid sequence” means a nucleic acid sequence corresponding to a reference nucleic acid sequence wherein the corresponding sequence encodes a peptide having substantially the same structure and function as the peptide encoded by the reference nucleic acid sequence; e.g., where only changes in amino acids not significantly affecting the peptide function occur. Preferably, the substantially similar nucleic acid sequence encodes the peptide encoded by the reference nucleic acid sequence. The percentage of identity between the substantially similar nucleic acid sequence and the reference nucleic acid sequence is at least about 50%, 60%, 70%, 80%, 85%, 90%, 95%, 99% or more. Substantial similarity of nucleic acid sequences can be determined by comparing the sequence identity of two sequences, for example by physical/chemical methods (e.g., hybridization) or by sequence alignment via computer algorithm. Suitable nucleic acid hybridization conditions to determine if a nucleotide sequence is substantially similar to a reference nucleotide sequence are: 7% sodium dodecyl sulfate SDS, 0.5 M NaPO₄, 1 mM EDTA at 50° C. with washing in 2× standard saline citrate (SSC), 0.1% SDS at 50° C.; preferably in 7% (SDS), 0.5 M NaPO₄, 1 mM EDTA at 50° C. with washing in 1×SSC, 0.1% SDS at 50° C.; preferably 7% SDS, 0.5 M NaPO₄, 1 mM EDTA at 50° C. with washing in 0.5×SSC, 0.1% SDS at 50° C.; and more preferably in 7% SDS, 0.5 M NaPO₄, 1 mM EDTA at 50° C. with washing in 0.1×SSC, 0.1% SDS at 65° C. Suitable computer algorithms to determine substantial similarity between two nucleic acid sequences include, GCS program package (Devereux et al., 1984 Nucl. Acids Res. 12:387), and the BLASTN or FASTA programs (Altschul et al., 1990 Proc. Natl. Acad. Sci. USA. 1990 87:14:5509-13; Altschul et al., J. Mol. Biol. 1990 215:3:403-10; Altschul et al., 1997 Nucleic Acids Res. 25:3389-3402). The default settings provided with these programs are suitable for determining substantial similarity of nucleic acid sequences for purposes of the present invention.

“Substantially purified” refers to a peptide or nucleic acid sequence which is substantially homogenous in character due to the removal of other compounds (e.g., other peptides, nucleic acids, carbohydrates, lipids) or other cells originally present. “Substantially purified” is not meant to exclude artificial or synthetic mixtures with other compounds, or the presence of impurities which do not interfere with biological activity, and which may be present, for example, due to incomplete purification, addition of stabilizers, or formulation into a pharmaceutically acceptable preparation.

As used herein, a “subunit” of a nucleic acid molecule is a nucleotide, and a “subunit” of a polypeptide is an amino acid.

“Synthetic mutant” includes any purposefully generated mutant or variant protein or nucleic acid. Such mutants can be generated by, for example, chemical mutagenesis, polymerase chain reaction (PCR) based approaches, or primer-based mutagenesis strategies well known to those skilled in the art.

The terms to “treat” or “treatment,” as used herein, refer to administering botulinum neurotoxin-neutralizing antibodies or compounds to reduce the frequency with which the effects or symptoms of a botulinum neurotoxin exposure or C. botulinum infection are experienced, to reduce the severity of symptoms, or to prevent effects or symptoms from occurring.

“Variant” as the term is used herein, is a nucleic acid sequence or a peptide sequence that differs in sequence from a reference nucleic acid sequence or peptide sequence respectively, but retains essential properties of the reference molecule. Changes in the sequence of a nucleic acid variant may not alter the amino acid sequence of a peptide encoded by the reference nucleic acid, or may result in amino acid substitutions, additions, deletions, fusions and truncations. Changes in the sequence of peptide variants are typically limited or conservative, so that the sequences of the reference peptide and the variant are closely similar overall and, in many regions, identical. A variant and reference peptide can differ in amino acid sequence by one or more substitutions, additions, deletions in any combination. A variant of a nucleic acid or peptide can be a naturally occurring such as an allelic variant, or can be a variant that is not known to occur naturally. Non-naturally occurring variants of nucleic acids and peptides may be made by mutagenesis techniques or by direct synthesis.

### DESCRIPTION OF THE FIGURES

FIG. 1, comprising FIGS. 1A and 1B, is a chart demonstrating the binding specificity of the botulinum neurotoxin 5A antibody for BoNT/A and BoNT/B as measured by a limiting dilution ELISA. FIG. 1A demonstrates the binding specificity of 5A IgM antibody and FIG. 1B demonstrates the binding specificity of 5A IgG antibody. The 5A IgG antibody was generated using recombinant DNA techniques and ectopic expression expressed in CHO cells.

FIG. 2 is a chart depicting a Western blot analysis demonstrating BoNT heavy chain binding by the botulinum neurotoxin 5A antibody.

FIG. 3 is a chart demonstrating the binding specificity of the botulinum neurotoxin 70A (IgM) antibody for BoNT/A as measured by a limiting dilution ELISA.

FIG. 4 is a chart demonstrating the binding specificity of the botulinum neurotoxin 50B (IgM) antibody for BoNT/B as measured by a limiting dilution ELISA.

FIG. 5, comprising FIGS. 5A through 5C, is a chart depicting the amino acid and nucleic acid sequences of the heavy and light chain variable domains of the botulinum neurotoxin 5A, 50B and 70A antibodies. FIGS. 5A-1 and 5A-2 show the amino acid and nucleic acid sequences of 5A heavy chain variable domain, respectively (SEQ ID NO:18 and SEQ ID NO:19). FIGS. 5A-3 and 5A-4 show the amino acid and nucleic acid sequences of 5A light chain variable domain, respectively (SEQ ID NO:20 and SEQ ID NO:21).

FIGS. 5B-1 and 5B-2 show the amino acid and nucleic acid sequences of 50B heavy chain variable domain, respectively (SEQ ID NO:22 and SEQ ID NO:23). FIGS. 5B-3 and 5B-4 show the amino acid and nucleic acid sequences of 50B light chain variable domain, respectively (SEQ ID NO:24 and SEQ ID NO:25).

FIGS. 5C-1 and 5C-2 show the amino acid and nucleic acid sequences of 70A heavy chain variable domain, respectively (SEQ ID NO:26 and SEQ ID NO:27). FIGS. 5C-3 and 5C-4 show the amino acid and nucleic acid sequences of 70A light chain variable domain, respectively (SEQ ID NO:28 and SEQ ID NO:29).

FIG. 6, comprising FIGS. 6A through 6C, is a chart depicting the amino acid and nucleic acid sequences of the heavy and light chain variable domains of the botulinum neurotoxin 5A, 50B and 70A antibodies. The light chain sequences shown do not contain the entirety of the first framework region of the variable domain. FIGS. 6A-1 and 6A-2 show the amino acid and nucleic acid sequences of 5A heavy chain variable domain including leader sequence residues (underlined), respectively (SEQ ID NO:30 and SEQ ID NO:31). FIGS. 6A-3 and 6A-4 show the amino acid and nucleic acid sequences of 5A light chain variable domain including some constant region residues (underlined), respectively (SEQ ID NO:32 and SEQ ID NO:33).

FIGS. 6B-1 and 6B-2 show the amino acid and nucleic acid sequences of 50B heavy chain variable domain including leader sequence residues (underlined), respectively (SEQ ID NO:34 and SEQ ID NO:35). FIGS. 6B-3 and 6B-4 show the amino acid and nucleic acid sequences of 50B light chain variable domain including some constant region residues (underlined), respectively (SEQ ID NO:36 and SEQ ID NO:37).

FIGS. 6C-1 and 6C-2 show the amino acid and nucleic acid sequences of 70A heavy chain variable domain including leader sequence residues (underlined), respectively (SEQ ID NO:38 and SEQ ID NO:39). FIGS. 6C-3 and 6C-4 show the amino acid and nucleic acid sequences of 70A light chain variable domain including some constant region residues (underlined), respectively (SEQ ID NO:40 and SEQ ID NO:41).

### DETAILED DESCRIPTION OF THE INVENTION

The present invention relates to novel antibodies that specifically bind botulinum neurotoxin. In one aspect, an antibody specifically binds to and neutralizes botulinum neurotoxin serotype A (BoNT/A), a neurotoxin produced by the anaerobic bacterium Clostridium botulinum. In another aspect, an antibody specifically binds to both botulinum neurotoxin serotype A and serotype B (BoNT/B). In yet another aspect, an antibody specifically binds to BoNT/B.

Neutralization, in this context, refers to a measurable decrease in the toxicity of the respective neurotoxin. Such a decrease in toxicity can be measured in vitro by a number of methods well known to those of skill in the art and those disclosed elsewhere herein. One such assay involves measuring the time to a given percentage (e.g. 50%) twitch tension reduction in a hemidiaphragm preparation.

Alternatively, toxicity can be determined in vivo. For example, one can measure the toxicity of BoNT/A and/or BoNT/B in a test animal (e.g. mouse) in the presence of one or more putative neutralizing antibodies. A neutralizing antibody can be combined with the botulinum neurotoxin prior to administration, or the animal can be administered the antibody prior to, simultaneous with, or after administration of the neurotoxin.

In terms of antibodies of the present invention able to neutralize BoNT/A, they are useful in the treatment of pathologies associated with botulinum neurotoxin poisoning. The treatments essentially comprise administering to the poisoned animal (e.g. human or non-human mammal) a quantity of BoNT/A neutralizing antibody sufficient to neutralize (e.g. mitigate or eliminate) symptoms of botulinum neurotoxin poisoning.

Such treatments are most desired and efficacious in acute cases, such as where vital capacity is less than 30-40 percent of predicted and/or paralysis is progressing rapidly and/or hypoxemia with absolute or relative hypercarbia is present. Treatment with a neutralizing antibody can be provided as a adjunct to other therapies (e.g. antibiotic treatment).

The antibodies of the present invention can also be used for the rapid detection/diagnosis of botulism including but not limited to type A toxin and type B toxin.

In another embodiment, the invention provides the epitopes specifically bound by BoNT/A and/or BoNT/B binding antibodies. These epitopes can be used to isolate, and/or identify and/or screen for other antibodies, including neutralizing antibodies, as described herein.

### Monoclonal Antibodies of the Invention

The invention provides monoclonal antibodies that specifically bind to botulinum neurotoxin (e.g. BoTN/A and/or BoTN/B). Antibodies of the present invention include those generated from a hybridoma cell line selected from the group consisting of the cell lines designated as 5-3-F11-2-F8-C2, 70A-D5, and 50B-B8, and recombinant antibodies comprising the heavy chain and/or light chain variable domain sequences encoded by the antibody genes expressed in each of the hybridomas. Each hybridoma secretes a monoclonal antibody, designated 5A, 70A, and 50B, respectively. The sequences of the heavy chain and light chain variable domains for each of these monoclonal antibodies has been determined and are disclosed herein.

Antibodies of the invention (5A, 50B and 70A) were generated against a botulinum neurotoxin (BoNT/A and/or BoNT/B). The human monoclonal antibodies of the present invention were produced using a SP2/mL-6 MPT2 cell line, which is a murine cell line that ectopically expresses murine interleukin-6 (mIL-6) and human telomerase (hTERT). This cell line has been demonstrated to efficiently produce human antibody-secreting hybridomas that are stable through multiple rounds of cloning. The SP2/mL-6 MPT2 cell line is discussed more fully elsewhere herein. Initially, the antibodies were of IgM isotype; however, as discussed elsewhere herein, the antibodies can be readily manipulated by recombinant methods in the art to generate an IgG isotype.

Hybridoma cell line 5-3-F11-2-F8-C2 produces the monoclonal antibody designated 5A. Antibody 5A comprises a heavy chain variable domain sequence as set forth in SEQ ID NO:18 and a light chain variable domain sequence as set forth in SEQ ID NO:20. SEQ ID NO:32, which comprises SEQ ID NO:20, is the amino acid sequence of the light chain variable domain including some constant region residues. In another aspect, the monoclonal antibody 5A comprises a heavy chain variable domain sequence and a light chain variable domain sequence encoded by a nucleic acid comprising the sequences as set forth in SEQ ID NO:19 and SEQ ID NO:21, respectively.

Hybridoma cell line 50B-B8 produces the monoclonal antibody designated 50B. Antibody 50B comprises a heavy chain variable domain sequence as set forth in SEQ ID NO:22 and a light chain variable domain sequence as set forth in SEQ ID NO:24. SEQ ID NO:36, which comprises SEQ ID NO:24, is the amino acid sequence of the light chain variable domain including some constant region residues. In another aspect, the monoclonal antibody 50B comprises a heavy chain variable domain sequence and a light chain variable domain sequence encoded by a nucleic acid comprising the sequences as set forth in SEQ ID NO:23 and SEQ ID NO:25, respectively.

Hybridoma cell line 70A-D5 produces the monoclonal antibody designated 70A. Antibody 70A comprises a heavy chain variable domain sequence as set forth in SEQ ID NO:26 and a light chain variable domain sequence as set forth in SEQ ID NO:28. SEQ ID NO:40, which comprises SEQ ID NO:28, is the amino acid sequence of the light chain variable domain including some constant region residues. In another aspect, the monoclonal antibody 70A comprises a heavy chain variable domain sequence and a light chain variable domain sequence encoded by a nucleic acid comprising the sequences as set forth in SEQ ID NO:27 and SEQ ID NO:29, respectively.

Botulinum neurotoxin antibodies (e.g. BoNT/A-neutralizing antibodies) of the invention include individual, allelic, strain, or species variants, and fragments thereof, both in their naturally occurring (full-length) forms and in recombinant forms.

### Recombinant Expression of Antibodies.

Antibodies of the present invention can be produced recombinantly using standard techniques well known to those of skill in the art. Typically, recombinant constructs comprise DNA segments encoding a complete human immunoglobulin heavy chain and/or a complete human immunoglobulin light chain of an immunoglobulin expressed by an antibody secreting cell line. Alternatively, DNA segments encoding only a portion of the primary antibody genes are produced, which portions possess binding and/or effector activities. Other recombinant constructs contain segments of the cell's immunoglobulin genes fused to segments of other immunoglobulin genes, particularly segments of other human constant region sequences (heavy and/or light chain).

Thus, in one embodiment, nucleic acid sequences coding for heavy and light chain variable domains of a monoclonal botulinum neurotoxin antibody disclosed herein, or a fragment or homolog thereof, are inserted into an appropriate expression vector. Alternatively, the heavy chain and light chain may be inserted into separate expression vectors. The expression vector comprises heavy and light chain constant regions, preferable human constant regions. Constant regions are obtained from human antibody-producing cells by standard cloning techniques. Alternatively, because genes representing the two classes of light chains and the five classes of heavy chains have been cloned, constant regions of human origin are readily available from these clones. The vector also contains the necessary elements for transcription and translation of the inserted protein-coding sequence so as to generate recombinant DNA molecules that direct the expression of heavy and light chain immunoglobulins for the formation of monoclonal botulinum neurotoxin neutralizing antibody.

Hybrid antibody binding fragments such as F(ab′)₂ and Fab fragments are prepared by designing a hybrid heavy chain gene in truncated form. For example, a hybrid gene encoding a F(ab′)₂ heavy chain portion would include DNA sequences encoding the CH₁ domain and hinge region of the heavy chain. Alternatively, such fragments can be obtained by enzymatic cleavage of a recombinant immunoglobulin. For instance, papain or pepsin cleavage can generate Fab or F(ab′)₂ fragments, respectively.

Using the sequence information provided herein, nucleic acids may be synthesized according to a number of standard methods known to those of skill in the art. Oligonucleotide synthesis, is preferably carried out on commercially available solid phase oligonucleotide synthesis machines or manually synthesized using the solid phase phosphoramidite triester method described by Beaucage et. al., 1981, Tetrahedron Letts. 22:1859-1862. The nucleic acid sequence may be optimized to reflect particular codon “preferences” for various expression systems according to standard methods well known to those of skill in the art.

Once a nucleic acid encoding a botulinum neurotoxin-binding antibody is synthesized, it may be amplified and/or cloned according to standard methods in order to produce recombinant antibodies of the invention. Molecular cloning techniques to achieve these ends are known in the art. A wide variety of cloning and in vitro amplification methods suitable for the construction of recombinant nucleic acids are known to those skilled in the art. Examples of these techniques and instructions sufficient to direct skilled artisan are found in Berger and Kimmel, Guide to Molecular Cloning Techniques, Methods in Enzymology volume 152 Academic Press, Inc., San Diego, Calif. (Berger); Sambrook et al., 1989 Molecular Cloning. A Laboratory Manual (2nd ed.) Vol. 1-3, Cold Spring Harbor Laboratory, Cold Spring Harbor Press, NY, (Sambrook); and Current Protocols in Molecular Biology, F. M. Ausubel et al., eds., Current Protocols, a joint venture between Greene Publishing Associates, Inc. and John Wiley & Sons, Inc., (1994 Supplement) (Ausubel). Methods of producing recombinant immunoglobulins are also known in the art. See, Cabilly, U.S. Pat. No. 4,816,567; and Queen et al., 1989 Proc. Nat'l Acad. Sci. USA 86:10029-10033.

Examples of techniques sufficient to direct persons of skill through in vitro amplification methods, including the polymerase chain reaction (PCR), the ligase chain reaction (LCR), and other DNA or RNA polymerase-mediated techniques are found in Berger, Sambrook, and Ausubel, as well as U.S. Pat. No. 4,683,202 and U.S. Pat. No. 5,426,039.

One may express the recombinant gene(s) in a variety of engineered cells known to those of skill in the art. Examples of such cells include bacteria, yeast, filamentous fungi, insect (especially employing baculoviral vectors), and mammalian cells. It is expected that those of skill in the art are knowledgeable in the numerous expression systems available for expression of botulinum neurotoxin-neutralizing antibodies.

Recombinant antibodies of the present invention include, but are not limited to, hybrid antibodies consisting of diverse components. For example, several different effector functions have been achieved by linking new sequences to those encoding the antigen binding domain within the antibody. In general, hybrid antibodies are produced by preparing, for each of the light and heavy chain components of the hybrid immunoglobulin, a fused gene comprising a first DNA segment that encodes at least the functional portion of the botulinum neurotoxin neutralizing antibody, such as a human variable region linked (e.g., functionally rearranged variable region with joining segment) to a second DNA segment encoding at least a part of a human constant region. Each fused gene is assembled in or inserted into an expression vector. Recipient cells capable of expressing the gene products are then transfected with the genes. The transfected recipient cells are cultured under conditions that permit expression of the incorporated genes and the expressed immunoglobulins or immunoglobulin chains are recovered.

Based on the present disclosure, a skilled artisan would recognize that the procedures used to produce hybrid antibodies includes the following steps (the order of some steps may be interchanged): (a) identifying and cloning the correct gene segment encoding the antigen binding portion of the antibody molecule; this gene segment (known as the VDJ, variable, diversity and joining regions for heavy chains or VJ, variable, joining regions for light chains (or simply as the V or variable region) may be in either the cDNA or genomic form; (b) cloning the gene segments encoding the constant region or desired part thereof; (c) ligating the variable region to the constant region so that the complete hybrid antibody is encoded in a transcribable and translatable form; (d) ligating this construct into a vector containing a selectable marker and gene control regions such as promoters, enhancers and poly(A) addition signals; (e) introducing the DNA into eukaryotic cells (transfection) such as mammalian lymphocytes; and culturing the host cell under conditions suitable for expression of the hybrid antibody.

Antibodies of several distinct antigen binding specificities have been manipulated by these protocols to produce hybrid proteins (e.g., anti-TNP: Boulianne et al., 1984, Nature 312:643; and anti-tumor antigens: Sahagan et al., 1986, J. Immunol. 137:1066). Likewise several different effector functions have been achieved by linking new sequences to those encoding the antigen binding region. Some of these include enzymes (Neuberger et al., 1984, Nature 312:604), immunoglobulin constant regions from another species and constant regions of another immunoglobulin chain (Sharon et al., 1984, Nature 309:364; and Tan et al., 1985, J. Immunol. 135:3565-3567).

In one preferred embodiment, a recombinant DNA vector is used to transfect a cell line that produces a BoNT/A and/or BoNT/B-binding antibody. The recombinant DNA vector contains a “replacement gene” to replace all or a portion of the gene encoding the immunoglobulin constant region in the cell line (e.g., a replacement gene may encode all or a portion of a constant region of a human immunoglobulin, a specific immunoglobulin class, or an enzyme, a toxin, a biologically active peptide, a growth factor, inhibitor, or a linker peptide to facilitate conjugation to a drug, toxin, or other molecule, etc.), and a “target sequence” which allows for targeted homologous recombination with immunoglobulin sequences within the antibody producing cell.

In another embodiment, a recombinant DNA vector is used to transfect a cell line that produces an antibody having a desired effector function, (e.g., a constant region of a human immunoglobulin or a constant region of another immunoglobulin chain). In this embodiment, the replacement gene contained in the recombinant vector may encode all or a portion of a region of an BoNT/A and/or BoNT/B-binding antibody and the target sequence contained in the recombinant vector allows for homologous recombination and targeted gene modification within the antibody producing cell. In either embodiment, when a portion of the variable or constant region is replaced, the resulting hybrid antibody may define the same antigen and/or have the same effector function yet be altered or improved so that the hybrid antibody may demonstrate a greater antigen specificity, greater affinity binding constant, increased effector function, or increased secretion and production by the transfected antibody producing cell line, etc.

Regardless of the embodiment practiced, the processes of selection for integrated DNA (via a selectable marker), screening for hybrid antibody production, and cell cloning, can be used to obtain a clone of cells producing the hybrid antibody.

Thus, a piece of DNA which encodes a modification for a monoclonal antibody can be targeted directly to the site of the expressed immunoglobulin gene within a B-cell or hybridoma cell line. DNA constructs for any particular modification may be used to alter the protein product of any monoclonal cell line or hybridoma. Such a procedure circumvents the costly and time consuming task of cloning both heavy and light chain variable region genes from each B-cell clone expressing a useful antigen specificity. In addition to circumventing the process of cloning variable region genes, the level of expression of the hybrid antibody should be higher when the gene is at its natural chromosomal location rather than at a random position.

Hybrid antibodies of the present invention can also be generated using genes encoding the variable region of immunoglobulin heavy and light chains from cells that produce a monoclonal botulinum neurotoxin antibody. For example, a hybridoma cell line that produces a desired monoclonal antibody against a botulinum neurotoxin provides a source of immunoglobulin variable region for the present hybrid antibodies. Methods of producing a hybridoma cell line producing a desired monoclonal antibody specific for an epitope recognized by one of antibodies 70A, 5A and 50B are described elsewhere herein.

Preferably, the fused genes encoding the heavy and light hybrid chains, or portions thereof, are assembled in one or two different expression vectors that can be used to cotransfect a recipient cell. In the case of using two different expression vectors, each vector contains two selectable genes, one for selection in a bacterial system and one for selection in a eukaryotic system, each vector having a different pair of genes. These vectors allow production and amplification of the fused genes in bacterial systems, and subsequent co-transfection of eukaryotic cells and selection of the cotransfected cells. Examples of selectable genes for the bacterial system include, but are not limited to, the genes that confer ampicillin resistance and the gene that confers chloramphenicol resistance. Two selectable genes for selection of eukaryotic transfectants are preferred, but are not limited to: (i) the xanthine-guanine phosphoribosyltransferase gene (gpt), and (ii) the phosphotransferase gene from Tn5 (designated neo). Selection with gpt is based on the ability of the enzyme encoded by this gene to use xanthine as a substrate for purine nucleotide synthesis; the analogous endogenous enzyme cannot. In a medium containing xanthine and mycophenolic acid, which blocks the conversion of inosine monophosphate to xanthine monophosphate, only cells expressing the gpt gene can survive. The product of the neo blocks the inhibition of protein synthesis in eukaryotic cells caused by the antibiotic G418 and other antibiotics of its class. The two selection procedures can be used simultaneously or sequentially to select for the expression of immunoglobulin chain genes introduced on two different DNA vectors into a eukaryotic cell.

The preferred recipient cell line for recombinant monoclonal antibody expression is a myeloma cell. Myeloma cells can synthesize, assemble and secrete immunoglobulins encoded by transfected immunoglobulin genes. Further, they possess the mechanism for glycosylation of the immunoglobulin. A particularly preferred recipient cell is a myeloma cell line that does not produce immunoglobulin, such as Sp2/0. These cell lines produce only the immunoglobulin encoded by the transfected immunoglobulin genes. Myeloma cells can be grown in culture or in the peritoneum of mice where secreted immunoglobulin can be obtained from ascites fluid. Other lymphoid cells such as B lymphocytes or hybridoma cells can serve as suitable recipient cells.

Several methods exist for transfecting lymphoid cells with vectors containing immunoglobulin encoding genes. A preferred way of introducing DNA into lymphoid cells is by electroporation. In this procedure recipient cells are subjected to an electric pulse in the presence of the DNA to be incorporated. Another way to introduce DNA is by protoplast fusion. In this method, lysozyme is used to strip cell walls from bacteria harboring the recombinant plasmid containing the immunoglobulin gene. The resulting spheroplasts are fused with myeloma cells with polyethylene glycol. After protoplast fusion, the transfectants are selected and isolated. Another technique that can be used to introduce DNA into many cell types is calcium phosphate precipitation.

The immunoglobulin genes can also be expressed in nonlymphoid cells, such as CHO cells, bacteria or yeast. When expressed in bacteria, the immunoglobulin heavy chains and light chains become part of inclusion bodies. Thus, the chains must be isolated and purified and then assembled into functional immunoglobulin molecules. Other strategies for expression in E. coli are available (see e.g., Pluckthun, A., 1991, BioTechnology 9:545-551; Skerra et al., 1991, BioTechnology 9:273-278), including secretion from E. coli as fusion proteins comprising a signal sequence.

### Vectors

The nucleic acids of the present invention may be replicated in wide variety of cloning vectors in a wide variety of host cells.

In brief summary, the expression of natural or synthetic nucleic acids encoding botulinum neurotoxin-binding antibodies is typically achieved by operably linking a nucleic acid encoding the antibody or portions thereof to a promoter, and incorporating the construct into an expression vector. The vectors are suitable for replication and integration in prokaryotes, eukaryotes, or both. Typical cloning vectors contain transcription and translation terminators, initiation sequences, and promoters useful for regulation of the expression of the desired nucleic acid sequence.

The nucleic acid can be cloned into a number of types of vectors. However, the present invention should not be construed to be limited to any particular vector. Instead, the present invention should be construed to encompass a wide plethora of vectors which are readily available and/or well-known in the art. For example, an the nucleic acid of the invention can be cloned into a vector including, but not limited to a plasmid, a phagemid, a phage derivative, an animal virus, and a cosmid. Vectors of particular interest include expression vectors, replication vectors, probe generation vectors, and sequencing vectors.

In specific embodiments, the expression vector is selected from the group consisting of a viral vector, a bacterial vector and a mammalian cell vector. Numerous expression vector systems exist that comprise at least a part or all of the compositions discussed above. Prokaryote- and/or eukaryote-vector based systems can be employed for use with the present invention to produce polynucleotides, or their cognate polypeptides. Many such systems are commercially and widely available.

Further, the expression vector may be provided to a cell, preferably a Chinese hamster ovary (CHO) cell in the form of a viral vector. Viral vector technology is well known in the art and is described, for example, in Sambrook et al. (1989), and in Ausubel et al. (1997), and in other virology and molecular biology manuals. Viruses, which are useful as vectors include, but are not limited to, retroviruses, adenoviruses, adeno-associated viruses, herpes viruses, and lentiviruses. Preferably, a murine stem cell virus (MSCV) vector is used to express a desired nucleic acid. MSCV vectors have been demonstrated to efficiently express desired nucleic acids in myeloma cells. However, the invention should not be limited to only using a MSCV vector, rather the immunoglobulin nucleic acids of the present invention can be introduced into cells using retroviral or other viral expression methods. Even more preferably, a Moloney Murine Leukemia Virus (MoMuLV) vector is used to express a desired nucleic acid in a cell such as CHO cells. In general, a suitable vector contains an origin of replication functional in at least one organism, a promoter sequence, convenient restriction endonuclease sites, and one or more selectable markers. (See, e.g., WO 01/96584; WO 01/29058; and U.S. Pat. No. 6,326,193.

For expression of the antibody or portions thereof, at least one module in each promoter functions to position the start site for RNA synthesis. The best known example of this is the TATA box, but in some promoters lacking a TATA box, such as the promoter for the mammalian terminal deoxynucleotidyl transferase gene and the promoter for the SV40 genes, a discrete element overlying the start site itself helps to fix the place of initiation.

Additional promoter elements, e.g., enhancers, regulate the frequency of transcriptional initiation. Typically, these are located in the region 30-110 by upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 by apart before activity begins to decline. Depending on the promoter, it appears that individual elements can function either co-operatively or independently to activate transcription.

A promoter may be one naturally associated with a gene or polynucleotide sequence, as may be obtained by isolating the 5′ non-coding sequences located upstream of the coding segment and/or exon. Such a promoter can be referred to as “endogenous.” Similarly, an enhancer may be one naturally associated with a polynucleotide sequence, located either downstream or upstream of that sequence. Alternatively, certain advantages will be gained by positioning the coding polynucleotide segment under the control of a recombinant or heterologous promoter, which refers to a promoter that is not normally associated with a polynucleotide sequence in its natural environment. A recombinant or heterologous enhancer refers also to an enhancer not normally associated with a polynucleotide sequence in its natural environment. Such promoters or enhancers may include promoters or enhancers of other genes, and promoters or enhancers isolated from any other prokaryotic, viral, or eukaryotic cell, and promoters or enhancers not “naturally occurring,” e.g., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR™, in connection with the compositions disclosed herein (U.S. Pat. No. 4,683,202, U.S. Pat. No. 5,928,906). Furthermore, it is contemplated the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the cell type, organelle, and organism chosen for expression. Those of skill in the art of molecular biology generally know how to use promoters, enhancers, and cell type combinations for protein expression, for example, see Sambrook et al. (1989). The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

An example of a promoter is the immediate early cytomegalovirus (CMV) promoter sequence. This promoter sequence is a strong constitutive promoter sequence capable of driving high levels of expression of any polynucleotide sequence operatively linked thereto. However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, Moloney virus promoter, the avian leukemia virus promoter, Epstein-Barr virus immediate early promoter, Rous sarcoma virus promoter, as well as human gene promoters such as, but not limited to, the actin promoter, the myosin promoter, the hemoglobin promoter, and the muscle creatine promoter. Further, the invention should not be limited to the use of constitutive promoters. Inducible promoters are also contemplated as part of the invention. The use of an inducible promoter in the invention provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter. Further, the invention includes the use of a tissue specific promoter, which promoter is active only in a desired tissue. Tissue specific promoters are well known in the art and include, but are not limited to, the HER-2 promoter and the PSA associated promoter sequences.

In order to assess the expression of an antibody or portions thereof, the expression vector to be introduced into a cell can also contain either a selectable marker gene or a reporter gene or both to facilitate identification and selection of expressing cells from the population of cells sought to be transfected or infected through viral vectors. In other embodiments, the selectable marker may be carried on a separate piece of DNA and used in a co-transfection procedure. Both selectable markers and reporter genes may be flanked with appropriate regulatory sequences to enable expression in the host cells. Useful selectable markers are known in the art and include, for example, antibiotic-resistance genes, such as neo and the like.

Reporter genes are used for identifying potentially transfected cells and for evaluating the functionality of regulatory sequences. Reporter genes that encode for easily assayable proteins are well known in the art. In general, a reporter gene is a gene that is not present in or expressed by the recipient organism or tissue and that encodes a protein whose expression is manifested by some easily detectable property, e.g., enzymatic activity. Expression of the reporter gene is assayed at a suitable time after the DNA has been introduced into the recipient cells.

Suitable reporter genes may include genes encoding luciferase, beta-galactosidase, chloramphenicol acetyl transferase, secreted alkaline phosphatase, or the green fluorescent protein gene (see, e.g., Ui-Tei et al., 2000 FEBS Lett. 479:79-82). Suitable expression systems are well known and may be prepared using well known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

Methods of introducing and expressing genes into a cell are known in the art. In the context of an expression vector, the vector can be readily introduced into a host cell, e.g., mammalian, bacterial, yeast or insect cell by any method in the art. For example, the expression vector can be transferred into a host cell by physical, chemical or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. and Ausubel et al.

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. A preferred colloidal system for use as a delivery vehicle in vitro and in vivo is a liposome (e.g., an artificial membrane vesicle). The preparation and use of such systems is well known in the art.

In the case where a non-viral delivery system is utilized, a preferred delivery vehicle is a liposome. The use of lipid formulations is contemplated for the introduction of the nucleic acids of the present invention, into a host cell (in vitro, ex vivo or in vivo). In a specific embodiment of the invention, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. The lipid, lipid/DNA or lipid/expression vector associated compositions of the present invention are not limited to any particular structure in solution. For example, they may be present in a bilayer structure, as micelles, or with a “collapsed” structure. They may also simply be interspersed in a solution, possibly forming aggregates which are not uniform in either size or shape.

Lipids are fatty substances which may be naturally occurring or synthetic lipids. For example, lipids include the fatty droplets that naturally occur in the cytoplasm as well as the class of compounds which are well known to those of skill in the art which contain long-chain aliphatic hydrocarbons and their derivatives, such as fatty acids, alcohols, amines, amino alcohols, and aldehydes.

Lipids suitable for use according to the present invention can be obtained from commercial sources. For example, dimyristyl phosphatidylcholine (“DMPC”) can be obtained from Sigma, St. Louis, Mo. Chemical Co., dicetyl phosphate (“DCP”) is obtained from K & K Laboratories (Plainview, N.Y.); cholesterol (“Chol”) is obtained from Calbiochem-Behring; dimyristyl phosphatidylglycerol (“DMPG”) and other lipids may be obtained from Avanti Polar Lipids, Inc. (Birmingham, Ala.). Stock solutions of lipids in chloroform or chloroform/methanol can be stored at about −20° C. Preferably, chloroform is used as the only solvent since it is more readily evaporated than methanol.

“Liposome” is a generic term encompassing a variety of single and multilamellar lipid vehicles formed by the generation of enclosed lipid bilayers or aggregates. Liposomes may be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). However, the present invention also encompasses compositions that have different structures in solution than the normal vesicular structure. For example, the lipids may assume a micellar structure or merely exist as nonuniform aggregates of lipid molecules. Also contemplated are lipofectamine-nucleic acid complexes.

Regardless of the method used to introduce exogenous nucleic acids into a host cell or otherwise expose a cell to the nucleic acid of the present invention, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention.

Following the generation of the antibodies or portions thereof of the present invention, the antibody or portions thereof can be used in a wide range of experimental and/or therapeutic purposes. For example, techniques for using and manipulating antibodies are found in Coligan (1991) Current Protocols in Immunology Wiley/Greene, NY; Harlow and Lane (1989) Antibodies: A Laboratory Manual Cold Spring Harbor Press, NY; Stites et al. (eds.) Basic and Clinical Immunology (4th ed.) Lange Medical Publications, Los Altos, Calif., and references cited therein; Goding (1986) Monoclonal Antibodies: Principles and Practice (2d ed.) Academic Press, New York, N.Y.; and Kohler and Milstein (1975) Nature 256: 495-497.

### Equivalents of Monoclonal Antibodies

The invention also includes functional equivalents of the antibodies described herein. Functional equivalents have binding characteristics comparable to those of the antibodies, and include, for example, hybridized and single chain antibodies, as well as fragments thereof. Methods of producing such functional equivalents are disclosed in PCT Application WO 93/21319 and PCT Application WO 89/09622.

Functional equivalents include polypeptides with amino acid sequences substantially the same as the amino acid sequence of the variable or hypervariable regions of the antibodies of the present invention. “Substantially the same” amino acid sequence is defined herein as a sequence with at least 70%, preferably at least about 80%, more preferably at least about 90%, even more preferably at least about 95%, and most preferably at least 99% homology to another amino acid sequence, as determined by the FASTA search method in accordance with Pearson and Lipman, 1988, Proc. Natl. Inst. Acad. Sci. USA 85:2444-2448. Hybrid antibodies of the present invention have constant regions derived substantially or exclusively from human antibody constant regions and variable regions derived substantially or exclusively from the sequence of the variable region of a monoclonal antibody from each stable hybridoma.

Single chain antibodies or Fv fragments are polypeptides that consist of the variable region of the heavy chain of the antibody linked to the variable region of the light chain, with or without an interconnecting linker. Thus, the Fv comprises an antibody combining site.

Functional equivalents of the antibodies of the invention further include fragments of antibodies that have the same, or substantially the same, binding characteristics to those of the whole antibody. Such fragments may contain one or both Fab fragments or the F(ab′)₂ fragment. Preferably the antibody fragments contain all six complement determining regions of the whole antibody, although fragments containing fewer than all of such regions, such as three, four or five complement determining regions, are also functional. The functional equivalents are members of the IgG immunoglobulin class and subclasses thereof, but may be or may combine any one of the following immunoglobulin classes: IgM, IgA, IgD, or IgE, and subclasses thereof. Heavy chains of various subclasses, such as the IgG subclasses, are responsible for different effector functions and thus, by choosing the desired heavy chain constant region, hybrid antibodies with desired effector function are produced. Preferred constant regions are gamma 1 (IgG1), gamma 2 (IgG2 and IgG), gamma 3 (IgG3) and gamma 4 (IgG4). The light chain constant region can be of the kappa or lambda type.

The immunoglobulins of the present invention can be monovalent, divalent or polyvalent. Monovalent immunoglobulins are dimers (HL) formed of a hybrid heavy chain associated through disulfide bridges with a hybrid light chain. Divalent immunoglobulins are tetramers (H₂L₂) formed of two dimers associated through at least one disulfide bridge.

### Modification of Antibodies

### A) Phage Display

A phage display can be used to increase antibody affinity. To create antibodies of higher affinity for a botulinum toxin, for instance a BoNT/A neutralizing antibody, mutant single chain variable fragment (scFv) gene repertories, based on the sequences disclosed herein can be created and expressed on the surface of phage. For a BoNT/A neutralizing antibody, mutant scFv gene repertories based on the variable domains of antibody 70A are prepared. Display of antibody fragments on the surface of viruses which infect bacteria (bacteriophage or phage) makes it possible to produce human or other mammalian antibodies with a wide range of affinities and kinetic characteristics. To display antibody fragments on the surface of phage (phage display), an antibody fragment gene is inserted into the gene encoding a phage surface protein and the antibody fragment-fusion protein is expressed on the phage surface (McCafferty et al., 1990, Nature 348: 552-554; Hoogenboom et al., 1991, Nucleic Acids Res. 19:4133-4137).

Since the antibody fragments on the surface of the phage are functional, those phage bearing antigen binding antibody fragments can be separated from non-binding or lower affinity phage by antigen affinity chromatography (McCafferty et al., 1990, Nature 348:552-554). Mixtures of phage are allowed to bind to the affinity matrix, non-binding or lower affinity phage are removed by washing, and bound phage are eluted by treatment with acid or alkali. Depending on the affinity of the antibody fragment, enrichment factors of 20 fold-1,000,000 fold are obtained by single round of affinity selection.

One approach for creating mutant scFv gene repertoires involves replacing either the V_{H} or V_{L} gene from a binding scFv with a repertoire of V_{H} or V_{L} genes (otherwise known as chain shuffling) (Clackson et al., 1991, Nature 352:624-628). Such gene repertoires contain numerous variable genes derived from the same germline gene as the binding scFv, but with point mutations (Marks et al., 1992, Biotechnology 10:779-783). Using light or heavy chain shuffling and phage display, the binding avidities of BoNT/A and/or BoNT/B-neutralizing antibody fragment can be dramatically increased.

In order to generate an antibody having an increased affinity, during the screening for the antibody, the antigen concentration is decreased in each round of selection, reaching a concentration less than the desired K_{d} by the final rounds of selection. This results in the selection of a desired antibody on the basis of affinity (Hawkins et al., 2002, J. Mol. Biol. 226: 889-896).

### B) Site Directed Mutagenesis

It is well known in the art that mutating amino acids that contact ligand has been shown to be an effective means of increasing the affinity of one protein molecule for its binding partner (Lowman et al., 1993, J. Mol. Biol. 234:564-578; Wells, 1990, Biochemistry 29:8509-8516). The majority of antigen-contacting, amino acid side chains in an antibody are located in the complementarity determining regions (CDRs). Three of the CDRs occur in the V_{H} (CDR1, CDR2, and CDR3) and three in the V_{L} (CDR1, CDR2, and CDR3) (Chothia et al., 1987, J. Mol. Biol. 196:901-917; Chothia et al., 1986, Science 233:755-8; Nhan et al., 1991, J. Mol. Biol. 217:133-151). These residues contribute the majority of binding energetics responsible for antibody affinity for antigen. Thus mutation of the CDRs and screening of the resulting mutants against BoNT/A and/or BoNT/A or the epitopes thereof identified herein, may be used to generate BoNT/A and/or BoNT/B-neutralizing antibodies having improved binding affinity to an epitope and/or bind with higher affinity to specific sub-serotypes (Smith et al., 2005, Infect. Immun. 73:5450-5457).

The CDRs are separated by framework regions. The framework regions spatially orient the CDR regions to shape the antigen-binding structure. Mutations to residues in either CDR regions or framework regions may alter and/or improve the binding characteristics of an antibody. Due to their structural role, changes to residues in framework regions may result in improperly folded antibody structures that may be inactive (Shlomchik et al, 1989, Prog Immunol. 7:415-423). Consequently, changes to framework region residues should be conservative changes and should preserve hydrophobic packing interactions and buried salt bridges. The determination of which amino acids in an immunoglobulin protein sequence contribute to which domains is well understood in the art and can be easily made using immunoglobulin gene analysis software, such as the software available from the Immunoenetics (IMGT) website (http://imgt(dot)cines(dot)fr). See also Lefranc et al., (2005, Nucleic Acids Res 33:D593-D597).

In a preferred embodiment, each CDR is randomized in a separate library. To simplify affinity measurements, existing antibodies or other lower affinity BoNT/A and/or BoNT/B-binding antibodies, are used as a template, rather than a higher affinity scFv. The CDR sequences of the highest affinity mutants from each CDR library are combined to obtain an additive increase in affinity. A similar approach has been used to increase the affinity of human growth hormone (hGH) for the growth hormone receptor over 1500 fold from 3.4×10⁻¹⁰ to 9.0×10⁻¹³ M (Lowman et al., 1993, J. Mol. Biol., 234:564-578).

To increase the affinity of BoNT/A and/or BoNT/B-binding antibodies, amino acid residues located in one or more CDRs (e.g. 9 amino acid residues located in V_{L} CDR3) are partially randomized by synthesizing a “doped” oligonucleotide in which the wild type nucleotide occurred with a frequency of about for example 49%. The oligonucleotide is used to amplify the remainder of the BoNT/A and/or BoNT/B-neutralizing scFv gene(s) using PCR.

For example, in one embodiment, to create a library in which V_{H} CDR3 is randomized, an oligonucleotide is synthesized which anneals to the BoNT/A and/or BoNT/B-binding antibody V_{H} framework 3 and encodes V_{H} CDR3 and a portion of framework 4. At the four positions to be randomized, the sequence “NNS” can be used, where N is any of the 4 nucleotides, and S is “C” or “T”. The oligonucleotide is used to amplify the BoNT/A-binding antibody V_{H} gene using PCR, creating a mutant BoNT/A and/or BoNT/B-binding antibody V_{H} gene repertoire. PCR is used to splice the V_{H} gene repertoire with the BoNT/A and/or BoNT/B-binding antibody light chain gene, and the resulting scFv gene repertoire is cloned into a phage display vector. Ligated vector DNA is used to transform electrocompetent E. coli to produce a phage antibody library.

To select higher affinity mutant scFv, each round of selection of the phage antibody libraries is conducted on decreasing amounts of BoNT/A and/or BoNT/B, as described elsewhere herein. Typically, 96 clones from the third and fourth round of selection are screened for binding to the BoNT/A and/or BoNTB antigen by ELISA on 96 well plates.

Other methods known in the art and used for mutagenizing antibodies include error-prone PCR, over-expression of dominant-negative mismatch repair proteins (WO 2004/046330), parsimonius mutagenesis (Razai et al., 2005, J Mol. Biol. 351:158-169) and chemical mutagenesis. See also: Chowdhury et al (2005, Methods 36:11-27) and Carter (2006, Nat Rev Immunol. 6:343-357). Identification of antibodies with desirable properties can be achieved using a variety of common screening methods (Hoogenboom, 2005, Nat. Biotechnol. 23:1105-1116).

C) Creation of Botulinum Neurotoxin-Binding (scFv′)₂ Homodimers

To create botulinum neurotoxin-binding (scFv′)₂ antibodies, two botulinum neurotoxin-binding scFvs are joined, either through a linker (e.g., a carbon linker, a peptide, etc.) or through a disulfide bond between, for example, two cysteines. Thus, for example, to create disulfide linked botulinum neurotoxin-binding scFv, a cysteine residue can be introduced by site directed mutagenesis.

In a particularly preferred embodiment, the (scFv′)₂ dimer is created by joining the scFv fragments through a linker, more preferably through a peptide linker. This can be accomplished by a wide variety of means well known to those of skill in the art. For example, one preferred approach is described by Holliger et al., 1993, Proc. Natl. Acad. Sci. USA 90:6444-6448 (see also WO 94/13804).

Typically, linkers are introduced by PCR cloning. For example, synthetic oligonucleotides encoding the linker can be used to PCR amplify the BoNT/A and/or BoNT/B-binding antibody V_{H} and V_{L} genes which are then spliced together to create the BoNT/A and/or BoNT/B-binding diabody gene. The gene is then cloned into an appropriate vector, expressed, and purified according to standard methods well known to those of skill in the art.

D) Preparation of Botulinum Neurotoxin-Binding (scFv)₂, Fab, and (Fab′)₂ Molecules

BoNT/A and/or BoNT/B-binding antibodies, such as a BoNT/A-neutralizing scFv, or variant(s) with higher affinity, are suitable templates for creating size and valency variants. For example, a BoNT/A-neutralizing (scFv′)₂ is created from a parent scFv derived from the variable domains of antibody 70A, as described above. An scFv gene can be excised using appropriate restriction enzymes and cloned into another vector.

A botulinum neurotoxin-binding Fab is expressed in E. coli using an expression vector similar to the one described by Better et. al., 1988, Science 240:1041-1043. To create a BoNT/A-binding Fab, the V_{H} and V_{L} genes are amplified from the scFv using PCR. The V_{H} gene is cloned into an expression vector (e.g., a PUC119 based bacterial expression vector) that provides an IgG C_{H1} domain downstream from, and in frame with, the V_{H} gene. The vector also contains a leader sequence to direct expressed V_{H}-C_{H1} domain into the periplasm, a leader sequence to direct expressed light chain into the periplasm, and cloning sites for the light chain gene. Clones containing the correct VH gene are identified, e.g., by PCR fingerprinting. The V_{L} gene is spliced to the C_{L} gene using PCR and cloned into the vector containing the V_{H}C_{H1} gene.

### Selection/Measurement of Neutralizing Antibodies

In preferred embodiments, selection of BoNT/A and/or BoNT/B-neutralizing antibodies (whether produced by phage display, immunization methods, hybridoma technology, etc.) involves screening the resulting antibodies for specific binding to an appropriate antigen. Preferably, the neutralizing antibodies of the present invention are selected to bind one or more epitopes bound by antibodies expressed by clones 5-3-F11-2-F8-C2, 70A-D5 and/or 50B-B8. Selection can be by any of a number of methods well known to those of skill in the art or disclosed elsewhere herein.

Selection for increased avidity involves measuring the affinity of a BoNT neutralizing antibody (or a modified BoNT-neutralizing antibody) for BoNT (or a BoNT fragment, or an epitope on BoNT, etc.). Methods of making such measurements are described elsewhere herein. In addition, the K_{d} of a BoNT/A-neutralizing antibody and the kinetics of binding to BoNT/A can be determined in a BIAcore, a biosensor based on surface plasmon resonance. For this technique, antigen is coupled to a derivatized sensor chip capable of detecting changes in mass. When antibody is passed over the sensor chip, antibody binds to the antigen resulting in an increase in mass that is quantifiable. Measurement of the rate of association as a function of antibody concentration can be used to calculate the association rate constant (kₒₙ). After the association phase, buffer is passed over the chip and the rate of dissociation of antibody (k_{off}) determined. Kₒₙ is typically measured in the range 1.0×10² to 5.0×10⁶ and k_{off} in the range 1.0×10⁻¹ to 1.0×10⁻⁶. The equilibrium constant IQ is then calculated as k_{off}/kₒₙ and thus is typically measured in the range 10⁻⁵ to 10⁻¹². Affinities measured in this manner correlate well with affinities measured in solution by fluorescence quench titration.

### Assaying for Cross-Reactivity at a Neutralizing Epitope

In a preferred embodiment, the antibodies of this invention specifically bind to one or more epitopes recognized by antibodies expressed by clones 5-3-F11-2-F8-C2, 70A-D5 and/or 50B-B8 disclosed herein. In other words, particularly preferred antibodies are cross-reactive with one of more of these antibodies. Means of assaying for cross-reactivity are well known to those of skill in the art (see, e.g., Dowbenko et al., 1998, J. Virol. 62: 4703-4711).

This can be ascertained by providing an isolated BoNT/A and/or BoNT/B polypeptide attached to a solid support and assaying the ability of a test antibody to compete with antibodies expressed by clones 5-3-F11-2-F8-C2, 70A-D5 and/or 50B-B8 disclosed herein for BoNT/A and BoNT/B binding. Thus, immunoassays in a competitive binding format are preferably used for crossreactivity determinations. For example, in one embodiment, a BoNT polypeptide is immobilized to a solid support. Antibodies to be tested added to the assay compete with antibodies expressed by clones 5-3-F11-2-F8-C2, 70A-D5 and/or 50B-B8 disclosed herein for the binding to the immobilized BoNT polypeptide. The ability of test antibodies to compete with the binding of the antibodies expressed by clones 5-3-F11-2-F8-C2, 70A-D5 and/or 50B-B8 disclosed herein to the immobilized protein are compared. The percent crossreactivity above proteins is then calculated, using standard calculations.

Cross-reactivity to antibodies expressed by clones 5-3-F11-2-F8-C2, 70A-D5 and/or 50B-B8 disclosed herein can be ascertained by a number of other standard techniques (see, e.g., Geysen et al., 1987, J. Immunol. Meth. 102:259-274). This technique involves the synthesis of large numbers of overlapping BoNT peptides. The synthesized peptides are then screened against one or more of the antibodies expressed by clones 5-3-F11-2-F8-C2, 70A-D5 and/or 50B-B8 disclosed herein and the characteristic epitopes specifically bound by these antibodies can be identified by binding specificity and affinity. The epitopes thus identified can be conveniently used for competitive assays as described herein to identify cross-reacting antibodies.

Epitope mapping can be conveniently prepared using “Multipin” peptide synthesis techniques (see, e.g., Geysen et al., 1987, Science 235:1184-1190). Using the known sequence of BoNT/A and/or BoNT/B, overlapping BoNT peptide sequences can be synthesized individually in a sequential manner on plastic pins in an array of one or more 96-well microtest plate(s).

The procedure for epitope mapping using this multipin peptide system is described in U.S. Pat. No. 5,739,306. Briefly, the pins are first treated with a pre-coat buffer containing 2% bovine serum albumin and 0.1% Tween 20 in PBS for 1 hour at room temperature. Then the pins are then inserted into the individual wells of 96-well microtest plate containing an antibody expressed by a hybridoma selected from the group consisting of 5-3-F11-2-F8-C2, 70A-D5 and 50B-B8. The incubation is preferably for about 1 hour at room temperature. The pins are washed in PBST (e.g., 3 rinses for every 10 minutes), and then incubated in the wells of a 96-well microtest plate containing 100 mu 1 of HRP-conjugated goat anti-mouse IgG (Fc) (Jackson ImmunoResearch Laboratories) at a 1:4,000 dilution for 1 hour at room temperature. After the pins are washed as before, the pins are placed into wells containing peroxidase substrate solution of diammonium 2,2′-azino-bis[3-ethylbenzthiazoline-b-sulfonate] (ABTS) and H₂O₂ (Kirkegaard & Perry Laboratories Inc., Gaithersburg, Md.) for 30 minutes at room temperature for color reaction. The plate is read at 405 nm by a plate reader (e.g., BioTek ELISA plate reader) against a background absorption wavelength of 492 nm. Wells showing color development indicated reactivity of the BoNT peptides in such wells with an antibody expressed by a hybridoma selected from the group consisting of 5-3-F11-2-F8-C2, 70A-D5 and 50B-B8.

Assaying for Neutralizing Activity of BoNT/A and/or BoNT/B Antibodies

Preferred antibodies of this invention act to neutralize (reduce or eliminate) the toxicity of botulinum neurotoxin (e.g. botulinum neurotoxin type A and/or type B). Neutralization can be evaluated in vivo or in vitro. In vivo neutralization measurements involve measuring changes in the lethality (e.g. LD₅₀ or other standard metric) due to a botulinum neurotoxin (e.g. botulinum neurotoxin type A and/or type B) administration due to the presence of one or more antibodies being tested for neutralizing activity. The neurotoxin can be directly administered to the test organism (e.g. mouse) or the organism can harbor a botulism infection (e.g., be infected with Clostridium botulinum). The antibody can be administered before, during, or after the injection of BoNT/A and/or BoNT/A neurotoxin or infection of the test animal. A decrease in the rate of progression, or mortality rate indicates that the antibody(s) have neutralizing activity.

A preferred in vitro assay for neutralizing activity uses a hemidiaphragm preparation (Deshpande et al., 1995, Toxicon 33:551-557). Briefly, purified antibodies are incubated with purified BoNT/A and/or BoNT/A for 30 minutes at room temperature and then added to the tissue bath, resulting in a final antibody concentration of about 2.0×10⁻⁸ M and a final neurotoxin concentration of about 2.0×10⁻¹¹ M. For each antibody studied, time to 50% twitch tension reduction is determined (e.g., three times for BoNT/A alone and three times for antibody plus BoNT/A). Differences between times to a given (arbitrary) percentage (e.g. 50%) twitch reduction are determined by standard statistical analyses (e.g. two-tailed t test) at standard levels of significance (e.g., a P value of <0.05 considered significant).

### Preparation of Monoclonal Antibodies

In certain embodiments, antibodies of the invention are selected to bind one or more epitopes bound by antibodies 70A, 50B and/or 5A disclosed herein. The antibodies can be raised in their native configurations or in non-native configurations. Anti-idiotypic antibodies can also be generated. Many methods of making antibodies that specifically bind to a particular epitope are known to those skilled in the art. The following discussion is presented as a general overview of the techniques available; however, a skilled artisan will recognize that many variations upon the following methods are known.

The epitopes to which each antibody of the invention binds can be identified using epitope mapping techniques known in the art. Non-limiting examples of epitope mapping techniques include binding assays using antigen mutants, antigen fragment binding assays, phage display of antigen peptides, MALDI mass spectroscopy and the “Multipin” peptide synthesis techniques described elsewhere herein. Once the epitope is identified, it can be used to generate additional monoclonal antibodies that specifically bind to it, using methods known in the art.

Summarized briefly, monoclonal antibody production proceeds by exposing a human or a non-human animal with an immunogen (e.g., the pentavalent botulinum neurotoxin vaccine). The animal is then sacrificed and the cells are taken from the spleen of the animal. Preferably, B-lymphocytes are obtained from peripheral blood (e.g. by phlebotomy) or other sites of the animal. These cells are then fused with myeloma cells. Preferably, the cells from the spleen are fused with the SP2/mL-6 MPT2 cell line. The result is a hybrid cell or “hybridoma” that is capable of reproducing in vitro. The population of hybridomas is then screened to isolate individual clones, each of which secrete a single antibody species to the immunogen. In this manner, the individual antibody species obtained are the products of immortalized and cloned single B cells from the immune animal generated in response to a specific site recognized on the immunogenic substance.

Alternative methods of immortalization include transformation with Epstein Barr Virus, oncogenes, or retroviruses, or other methods known in the art. Colonies arising from single immortalized cells are screened for production of antibodies of the desired specificity and affinity for the BoNT/A and/or BoNT/B antigen, and yield of the monoclonal antibodies produced by such cells is enhanced by various techniques, including injection into the peritoneal cavity of a vertebrate (preferably mammalian) host. The antibodies of the present invention are used with or without modification, and include hybrid antibodies such as antibodies having different antibody isotypes.

In some instances, it is desirable to prepare monoclonal antibodies from various mammalian hosts, such as mice, rodents, primates, humans, etc. Descriptions of techniques for preparing such monoclonal antibodies are found in, e.g., Stites et al. (eds.) Basic and Clinical Immunology (4th ed.) Lange Medical Publications, Los Altos, Calif., and references cited therein; Harlow and Lane, supra; Goding (1986) Monoclonal Antibodies: Principles and Practice (2d ed.) Academic Press, New York, N.Y.; and Kohler and Milstein (1975) Nature 256: 495-497.

As more fully discussed elsewhere herein, the antibodies of the present invention can be manipulated, for example, by way of mutating the sequences corresponding to the antibodies described herein to create antibodies with altered binding characteristics. For example, a cell expressing a specific antibody gene sequence can be subjected to a mutagenesis procedure such that a progeny of that cell produces antibodies with different characteristics than the original antibody. Preferably, the antibodies produced from the progeny have an increased avidity for the botulinum neurotoxin compared to the original antibody.

SP2/mL-6 MPT2 Cell Line for Generating Human Antibodies

The invention includes a fully human anti-BoNT/A and/or anti-BoNT/B-binding antibody. Human antibodies consist entirely of characteristically human polypeptide sequences. The human BoNT/A and/or BoNT/B-binding antibodies of the invention can be produced in using a wide variety of methods (see, e.g., U.S. Pat. No. 5,001,065 and WO 03/052082).

In one embodiment, human BoNT/A and/or BoNT/B-binding antibodies of the present invention are initially produced by a type of hybridoma or hybrid cell termed a trioma. However, any hybridoma can be used to produce the antibodies of the present invention. Genes encoding the antibodies are then cloned and expressed in other cells, particularly, nonhuman mammalian cells. The general approach for producing human antibodies by trioma technology has been described by Ostberg et al., 1983, Hybridoma 2:361-367, Ostberg, U.S. Pat. No. 4,634,664, and Engelman et al., U.S. Pat. No. 4,634,666. The antibody-producing cell lines obtained by this method are called triomas because they are descended from three cells; two human and one mouse. However, the antibodies of the invention are preferably produced from a type of hybridoma or trioma that is formed by ectopically expressing phenotype altering gene(s). In a preferred embodiment, two or more genes are expressed in a cell and therefore the cell is useful as a fusion partner cell line to produce a hybrid/hybridoma that produces the desired antibody (see, e.g., WO 03/052082). For example, the two genes that can be expressed in such a cell can be murine interleukin-6 (mIL-6) and human telomerse (hTERT). This cell which has been modified to express mIL-6 and hTERT can then be fused with a primary human B-cell, to generate a type of hybridoma that produces antibodies of the present invention.

In a preferred embodiment of the invention, the antibody-producing cell is generated by fusing the cell line, SP2/mL-6 MPT, with a primary human B-cell. The SP2/mL-6 MPT cell line is a modified SP2/0 cell line, in which the genes mIL-6 and hTERT have been ectopically expressed. The SP2/0 cell line is itself a hybridoma, formed by fusion of a murine myeloma cell with a primary murine B-cell (Shulman et al., 1978, Nature 276:269-270). The hybridomas of this embodiment are thus triomas, derived from a fusion of two murine cells and one human cell. For example, the antibody-producing cell can be generated using splenic B-lymphocytes from a human or a non-human animal immunized with a botulinum neurotoxin based immunogen, by fusing the splenic B-lymphocytes with the SP2/mL-6 MPT cell line.

Also encompassed in the present invention are antibodies generated using the methods disclosed herein, wherein B-lymphocytes are isolated from a human or non-human animal immunized against a BoNT polypeptide (e.g., BoNT/A, BoNT/B, or BoNT subsequences including, but not limited to epitopes specifically bound by antibodies expressed by clones 5-3-F11-2-F8-C2, 70A-D5 and/or 50B-B8 disclosed herein). The B-lymphocytes are obtained from the spleen, blood or lymph nodes of human donor. If antibodies against a specific antigen or epitope are desired, it is preferable to use that antigen or epitope thereof as the immunogen rather than the entire polypeptide. Alternatively, B-lymphocytes are obtained from an unimmunized individual and stimulated with a BoNT/A and/or BoNT/A polypeptide, or a epitope thereof, in vitro. In a further variation, B-lymphocytes are obtained from an infected, or otherwise immunized individual, and then hyperimmunized by exposure to a BoNT/A and/or BoNT/B polypeptide for about seven to fourteen days, in vitro.

The immunized B-lymphocytes prepared by one of the above procedures can be fused with the SP2/mL-6 MPT cell line by well known methods. For example, the cells are treated with 40-50% polyethylene glycol of MW 1000-4000, at about 37° C. for about 5-10 minutes. Cells are separated from the fusion mixture and propagated in media selective for the desired hybrids. When the hybrid cell is resistant to 8-azaguanine, the cell is conveniently selected by successive passaging of the cell on HAT or AH medium. Other selective procedures can be used depending on the nature of the cells used in fusion. Clones secreting antibodies having the required binding specificity are identified by assaying the antibody secreted into the culture medium for the ability to bind to the botulinum neurotoxin polypeptide or an epitope thereof. The antibody producing cells having the desired specificity are subcloned by the limiting dilution technique and grown in vitro in culture medium, or are injected into selected host animals and grown in vivo.

The antibodies produced from the cell lines are then tested for the ability to bind a BoNT/A and/or BoNT/B polypeptide or an epitope thereof. Antibodies are also tested for the capacity to neutralize BoNT/A and/or BoNT/B neurotoxin. Antibodies are separated from the resulting culture medium or body fluids by conventional antibody-fractionation procedures, such as ammonium sulfate precipitation, DEAE cellulose chromatography and affinity chromatography.

In addition to obtaining botulinum neurotoxin-binding antibodies from a hybridoma cell, the antibodies can also be generated by cloning antibody genes from the hybridoma into one or more expression vectors, and transforming the vector into a cell line such as the cell lines typically used for expression of recombinant or humanized immunoglobulins.

The genes encoding the heavy and light chains of immunoglobulins secreted by the cell lines are cloned according to methods, including but not limited to, the polymerase chain reaction (PCR), known in the art (see, e.g., Sambrook et al., Molecular Cloning: A Laboratory Manual, 2nd ed., Cold Spring Harbor, N.Y., 1989; Berger & Kimmel, Methods in Enzymology, Vol. 152: Guide to Molecular Cloning Techniques, Academic Press, Inc., San Diego, Calif., 1987; Co et al., 1992, J. Immunol. 148:1149). For example, genes encoding heavy and light chains are cloned from the antibody secreting cell's genomic DNA or cDNA is produced by reverse transcription of the cell's RNA. Cloning is accomplished by conventional techniques including the use of PCR primers that hybridize to the sequences flanking or overlapping the genes, or segments of genes, to be cloned.

In addition to the DNA segments encoding BoNT/A and/or BoNT/B-binding immunoglobulins or fragments thereof, other substantially homologous modified immunoglobulins can be readily designed and manufactured utilizing various recombinant DNA techniques known to those skilled in the art such as site-directed mutagenesis. Such modified segments will usually retain antigen binding capacity and/or effector function. Moreover, the modified segments are usually not so far changed from the original genomic sequences of the antibody producing cell to prevent hybridization to these sequences under stringent conditions. Because, like many genes, immunoglobulin genes contain separate functional regions, each having one or more distinct biological activities, the genes may be fused to functional regions from other genes to produce fusion proteins (e.g., immunotoxins) having novel properties or novel combinations of properties.

Other approaches to antibody production include in vitro immunization of human blood. In this approach, human blood lymphocytes capable of producing human antibodies are produced. Human peripheral blood is collected from the subject and is treated to recover mononuclear cells. The suppressor T-cells are the removed from the cell mixture and the remaining cells are suspended in a tissue culture medium. Antigen and autologous serum is then added to the culture. Preferably a nonspecific lymphocyte activator is also added to the culture. The cells are then incubated for a period of time so that they produce the specific desired antibody. The cells can then be fused to a human myeloma cell to immortalize the cell line, thereby to permit continuous production of the antibody (see U.S. Pat. No. 4,716,111).

In a preferred embodiment, antibodies of the present invention are produced using a mouse-human hybridoma which produces the desired botulinum-neutralizing antibody. Other approaches include immunization of murines transformed to express human immunoglobulin genes, and phage display screening (Vaughan et al. supra.).

### Therapeutic Use and Pharmaceutical Compositions

One skilled in the art can readily determine an effective amount of botulinum neurotoxin-neutralizing antibodies to be administered to a given subject, by taking into account factors such as the size and weight of the subject; the extent of disease penetration; the age, health and sex of the subject; the route of administration; and whether the administration is regional or systemic. Generally, the amount of antibody administered to a subject depends upon the amount of botulinum neurotoxin that needs to be neutralized and the amount of botulinum neurotoxin-neutralizing activity exhibited by the antibodies. Those skilled in the art may derive appropriate dosages and schedules of administration to suit the specific circumstances and needs of the subject. For example, suitable doses of each antibody to be administered can be estimated from the amount of botulinum neurotoxin to which a subject has been exposed, or the amount of botulinum neurotoxin to which the subject is in risk of being exposed. Typically, dosages of antibody are between about 0.001 mg/kg and about 100 mg/kg body weight. In some embodiments, dosages are between about 0.01 mg/kg and about 60 mg/kg body weight.

It is understood that the effective dosage will depend on the age, sex, health, and weight of the recipient, kind of concurrent treatment, if any, frequency of treatment, and the nature of the effect desired. The most preferred dosage will be tailored to the individual subject, as is understood and determinable by one of skill in the art, without undue experimentation.

A mixture of botulinum neurotoxin-neutralizing human antibodies can be administered in equimolar concentrations to a subject in need of such treatment. In another instance, the antibodies are administered in concentrations which are not equimolar. In other instances, the antibodies are administered as equal amounts of protein, by weight, per kilogram of body weight. For example, the antibodies can be administered in equal amounts, based on the weight of the subject. In another instance, the antibodies are administered in unequal amounts. In yet other instances, the amount of each antibody to be administered is based on its neutralizing activity. For example, a mixture with between about 1 IU/kg body weight and about 50 IU/kg body weight of botulinum neurotoxin-neutralizing activity can be administered.

In general, the schedule or timing of administration of a mixture of botulinum neurotoxin-neutralizing human antibodies is according to the accepted practice for the procedure being performed.

When used in vivo, the antibodies, either in their native form and/or in a recombinant form, are preferably administered as a pharmaceutical composition, comprising a mixture, and a pharmaceutically acceptable carrier. The antibodies may be present in a pharmaceutical composition in an amount from 0.001 to 99.9 wt %, more preferably from about 0.01 to 99.0 wt %, and even more preferably from 0.1 to 50 wt %. To achieve good plasma concentrations, an antibody, or a combination of antibodies, may be administered, for example, by intravenous injection, as a solution comprising 0.1 to 1.0% of the active agent.

All of the different recombinant botulinum neurotoxin-neutralizing human antibodies to be administered need not be administered together in a single composition. The different botulinum neurotoxin-neutralizing antibody can be administered in separate compositions. For example, if three different antibodies are to be administered, the three different antibodies can be delivered in three separate compositions. In addition, each antibody can be delivered at the same time, or the antibodies can be delivered consecutively with respect to one another. Thus, the mixture of botulinum neurotoxin-neutralizing antibodies can be administered in a single composition, or in multiple compositions comprising one or more recombinant botulinum neurotoxin-neutralizing antibodies.

The recombinant botulinum neurotoxin-neutralizing human antibodies, or pharmaceutical compositions comprising these compounds, may be administered by any method designed to allow compounds to have a physiological effect. Administration may occur enterally or parenterally; for example orally, rectally, intracisternally, intravaginally, intraperitoneally, locally (e.g., with powders, ointments or drops). Parenteral administration is preferred. Particularly preferred parenteral administration methods include intravascular administration (e.g., intravenous bolus injection, intravenous infusion, intra-arterial bolus injection, intra-arterial infusion and catheter instillation into the vasculature), peri- and intra-target tissue injection, subcutaneous injection or deposition including subcutaneous infusion (such as by osmotic pumps), intramuscular injection, intraperitoneal injection, and direct application to the target area, for example by a catheter or other placement device.

The botulinum neurotoxin-neutralizing antibodies of the present invention are useful for prophylactic and/or therapeutic treatment. The pharmaceutical compositions can be administered in a variety of unit dosage forms depending upon the method of administration. For example, unit dosage forms suitable for oral administration include powder, tablets, pills, capsules and lozenges. It is recognized that the fusion proteins and pharmaceutical compositions of this invention, when administered orally, must be protected from digestion. This is typically accomplished either by complexing the protein with a composition to render it resistant to acidic and enzymatic hydrolysis or by packaging the protein in an appropriately resistant carrier such as a liposome. Means of protecting proteins from digestion are well known in the art.

The pharmaceutical compositions of this invention are particularly useful for parenteral administration, such as intravenous administration or administration into a body cavity or lumen of an organ. The compositions for administration will commonly comprise a solution of the BoNT/A and/or BoNT/B-neutralizing antibody dissolved in a pharmaceutically acceptable carrier, preferably an aqueous carrier. A variety of aqueous carriers can be used, e.g., buffered saline and the like. These solutions are sterile and generally free of undesirable matter. These compositions may be sterilized by conventional, well known sterilization techniques. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of BoNT/A-neutralizing antibody in these formulations can vary widely, and will be selected primarily based on fluid volumes, viscosities, body weight and the like in accordance with the particular mode of administration selected and the subject's needs.

Thus, a typical pharmaceutical composition for intravenous administration would be about 0.1 to 10 mg per subject per day. Dosages from 0.1 up to about 100 mg per subject per day may be used, particularly when the drug is administered to a secluded site and not into the blood stream, such as into a body cavity or into a lumen of an organ. Methods for preparing parenterally administrable compositions will be known or apparent to those skilled in the art and are described in more detail in such publications as Remington's Pharmaceutical Science, 15th ed., Mack Publishing Company, Easton, Pa. (1980).

The compositions containing the antibody of the present invention, fusion proteins or a cocktail thereof (e.g., with other proteins, including but not limited to other antibodies that bind botulinum neurotoxins) can be administered for therapeutic treatments. In therapeutic applications, preferred pharmaceutical compositions are administered in a dosage sufficient to neutralize (mitigate or eliminate) BoNT/A and/or BoNT/B toxin (e.g., reduce or eliminate a symptom of BoNT/A and/or BoNT/B poisoning (botulism)). An amount adequate to accomplish this is defined as a “therapeutically effective dose.” Amounts effective for this use will depend upon the severity of the disease and the general state of the subject's health.

Single or multiple administrations of the compositions may be administered depending on the dosage and frequency as required and tolerated by the subject. In any event, the composition should provide a sufficient quantity of the proteins of this invention to effectively treat the subject.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Pharmaceutical compositions that are useful in the methods of the invention may be prepared, packaged, or sold in formulations suitable for oral, rectal, vaginal, parenteral, topical, pulmonary, intranasal, buccal, ophthalmic, or another route of administration. Other contemplated formulations include projected nanoparticles, liposomal preparations, resealed erythrocytes containing the active ingredient, and immunologically-based formulations.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the invention may further comprise one or more additional pharmaceutically active agents. Particularly contemplated additional agents include anti-emetics and scavengers such as cyanide and cyanate scavengers and AZT, protease inhibitors, reverse transcriptase inhibitors, interleukin-2, interferons, cytokines, and the like.

Controlled- or sustained-release formulations of a pharmaceutical composition of the invention may be made using conventional technology.

A formulation of a pharmaceutical composition of the invention suitable for oral administration may be prepared, packaged, or sold in the form of a discrete solid dose unit including, but not limited to, a tablet, a hard or soft capsule, a cachet, a troche, or a lozenge, each containing a predetermined amount of the active ingredient. Other formulations suitable for oral administration include, but are not limited to, a powdered or granular formulation, an aqueous or oily suspension, an aqueous or oily solution, or an emulsion.

As used herein, an “oily” liquid is one which comprises a carbon-containing liquid molecule and which exhibits a less polar character than water.

A tablet comprising the active ingredient may, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface active agent, and a dispersing agent. Molded tablets may be made by molding, in a suitable device, a mixture of the active ingredient, a pharmaceutically acceptable carrier, and at least sufficient liquid to moisten the mixture.

Pharmaceutically acceptable excipients used in the manufacture of tablets include, but are not limited to, inert diluents, granulating and disintegrating agents, binding agents, and lubricating agents. Known dispersing agents include, but are not limited to, potato starch and sodium starch glycolate. Known surface active agents include, but are not limited to, sodium lauryl sulphate. Known diluents include, but are not limited to, calcium carbonate, sodium carbonate, lactose, microcrystalline cellulose, calcium phosphate, calcium hydrogen phosphate, and sodium phosphate. Known granulating and disintegrating agents include, but are not limited to, corn starch and alginic acid. Known binding agents include, but are not limited to, gelatin, acacia, pre-gelatinized maize starch, polyvinylpyrrolidone, and hydroxypropyl methylcellulose. Known lubricating agents include, but are not limited to, magnesium stearate, stearic acid, silica, and talc.

Tablets may be non-coated or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and 4,265,874 to form osmotically-controlled release tablets. Tablets may further comprise a sweetening agent, a flavoring agent, a coloring agent, a preservative, or some combination of these in order to provide pharmaceutically elegant and palatable preparation.

Hard capsules comprising the active ingredient may be made using a physiologically degradable composition, such as gelatin. Such hard capsules comprise the active ingredient, and may further comprise additional ingredients including, for example, an inert solid diluent such as calcium carbonate, calcium phosphate, or kaolin.

Soft gelatin capsules comprising the active ingredient may be made using a physiologically degradable composition, such as gelatin. Such soft capsules comprise the active ingredient, which may be mixed with water or an oil medium such as peanut oil, liquid paraffin, or olive oil.

Liquid formulations of a pharmaceutical composition of the invention which are suitable for oral administration may be prepared, packaged, and sold either in liquid form or in the form of a dry product intended for reconstitution with water or another suitable vehicle prior to use.

Liquid suspensions may be prepared using conventional methods to achieve suspension of the active ingredient in an aqueous or oily vehicle. Aqueous vehicles include, for example, water and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropyl methylcellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g., polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin and acacia. Known preservatives include, but are not limited to, methyl, ethyl, or n-propyl-para-hydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

Liquid solutions of the active ingredient in aqueous or oily solvents may be prepared in substantially the same manner as liquid suspensions, the primary difference being that the active ingredient is dissolved, rather than suspended in the solvent. Liquid solutions of the pharmaceutical composition of the invention may comprise each of the components described with regard to liquid suspensions, it being understood that suspending agents will not necessarily aid dissolution of the active ingredient in the solvent. Aqueous solvents include, for example, water and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the invention may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, and a preservative. Additional excipients, such as fillers and sweetening, flavoring, or coloring agents, may also be included in these formulations.

A pharmaceutical composition of the invention may also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

Methods for impregnating or coating a material with a chemical composition are known in the art, and include, but are not limited to methods of depositing or binding a chemical composition onto a surface, methods of incorporating a chemical composition into the structure of a material during the synthesis of the material (e.g. such as with a physiologically degradable material), and methods of absorbing an aqueous or oily solution or suspension into an absorbent material, with or without subsequent drying.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (e.g. powder or granular) form for reconstitution with a suitable vehicle (e.g. sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

A pharmaceutical composition of the invention may be prepared, packaged, or sold in a formulation suitable for pulmonary administration via the buccal cavity. Such a formulation may comprise dry particles which comprise the active ingredient and which have a diameter in the range from about 0.5 to about 7 nanometers, and preferably from about 1 to about 6 nanometers. Such compositions are conveniently in the form of dry powders for administration using a device comprising a dry powder reservoir to which a stream of propellant may be directed to disperse the powder or using a self-propelling solvent/powder-dispensing container such as a device comprising the active ingredient dissolved or suspended in a low-boiling propellant in a sealed container. Preferably, such powders comprise particles wherein at least 98% of the particles by weight have a diameter greater than 0.5 nanometers and at least 95% of the particles by number have a diameter less than 7 nanometers. More preferably, at least 95% of the particles by weight have a diameter greater than 1 nanometer and at least 90% of the particles by number have a diameter less than 6 nanometers. Dry powder compositions preferably include a solid fine powder diluent such as sugar and are conveniently provided in a unit dose form.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Remington's Pharmaceutical Sciences (1985, Genaro, ed., Mack Publishing Co., Easton, Pa.), which is incorporated herein by reference.

### Diagnostic Assays

The BoNT/A and/or BoNT/B-binding antibodies of the present invention may be used for the in vivo or in vitro detection of BoNT/A and/or BoNT/B toxin and thus, are useful in the diagnosis (e.g. confirmatory diagnosis) of botulism. The detection and/or quantification of BoNT/A and/or BoNT/B in a biological sample obtained from an organism is indicative of a Clostridium botulinum infection of that organism.

For example, a BoNT/A antigen may be quantified in a biological sample derived from a subject such as a cell, or a tissue sample derived from a subject. As used herein, a biological sample is a sample of biological tissue or fluid that contains a BoNT/A and/or BoNT/B concentration that may be correlated with and indicative of a Clostridium botulinum infection. Preferred biological samples include blood, urine, saliva, and tissue biopsies.

Although the sample is typically taken from a human subject, the assays can be used to detect BoNT/A and/or BoNT/B antigen in cells from mammals in general, such as dogs, cats, sheep, cattle and pigs, and most particularly primates such as humans, chimpanzees, gorillas, macaques, and baboons, and rodents such as mice, rats, and guinea pigs.

Tissue or fluid samples are isolated from a subject according to standard methods well known to those of skill in the art, most typically by biopsy. The sample is optionally pretreated as necessary by dilution in an appropriate buffer solution or concentrated, if desired. Any of a number of standard aqueous buffer solutions, employing one of a variety of buffers, such as phosphate, Tris, or the like, at physiological pH can be used.

### Immunological Binding Assays

The BoNT/A and/or BoNT/B polypeptide is preferably detected in an immunoassay utilizing a BoNT/A and/or BoNT/B-neutralizing antibody as a capture agent that specifically binds to the BoNT/A and/or BoNT/B polypeptide. As used herein, an immunoassay is an assay that utilizes an antibody (e.g. a BoNT/A-neutralizing antibody) to specifically bind an analyte (e.g., BoNT/A). The immunoassay is characterized by the use of specific antibody binding to a BoNT/A-neutralizing antibody as opposed to other physical or chemical properties to isolate, target, and quantify the BoNT/A analyte. The BoNT/A marker may be detected and quantified using any of a number of well recognized immunological binding assays (See for example, U.S. Pat. Nos. 4,366,241; 4,376,110; 4,517,288; and 4,837,168).

The immunoassays of the present invention are performed in any of several configurations, e.g., those reviewed in Maggio (ed.) (1980) Enzyme Immunoassay CRC Press, Boca Raton, Fla.; Tijan (1985) “Practice and Theory of Enzyme Immunoassays,” Laboratory Techniques in Biochemistry and Molecular Biology, Elsevier Science Publishers B.V., Amsterdam; Harlow and Lane, supra; Chan (ed.) (1987) Immunoassay: A Practical Guide Academic Press, Orlando, Fla.; Price and Newman (eds.) (1991) Principles and Practice of Immunoassays Stockton Press, NY; and Ngo (ed.) (1988) Non isotopic Immunoassays Plenum Press, NY. Immunoassays often utilize a labeling agent to specifically bind to and label the binding complex formed by the capture agent and the analyte (e.g., a BoNT/A-neutralizing antibody/BoNT/A complex). The labeling agent may itself be one of the moieties comprising the antibody/analyte complex. Thus, the labeling agent may be a labeled BoNT/A or a labeled BoNT/A-neutralizing antibody. Alternatively, the labeling agent is optionally a third moiety, such as another antibody, that specifically binds to the BoNT/A-neutralizing antibody, the BoNT/A peptide, the anti-body/polypeptide complex, or to a modified capture group (e.g., biotin) which is covalently linked to BoNT/A or to the BoNT/A-neutralizing antibody.

In one embodiment, the labeling agent is an antibody that specifically binds to the BoNT/A and/or BoNT/B-neutralizing antibody. Such agents are well known to those of skill in the art, and most typically comprise labeled antibodies that specifically bind antibodies of the particular animal species from which the BoNT/A and/or BoNT/B-neutralizing antibody is derived (e.g., an anti-species antibody). Thus, for example, where the capture agent is a human derived BoNT/A and/or BoNT/B-neutralizing antibody, the label agent may be a mouse anti-human IgG, e.g., an antibody specific to the constant region of the human antibody.

Other proteins capable of specifically binding immunoglobulin constant regions, such as streptococcal protein A or protein G are also used as the labeling agent. These proteins are normal constituents of the cell walls of streptococcal bacteria. They exhibit a strong non immunogenic reactivity with immunoglobulin constant regions from a variety of species.

Throughout the assays, incubation and/or washing steps may be required after each combination of reagents. Incubation steps can vary from about 5 seconds to several hours, preferably from about 5 minutes to about 24 hours. However, the incubation time will depend upon the assay format, analyte, volume of solution, concentrations, and the like. Usually, the assays are carried out at ambient temperature, although they can be conducted over a range of temperatures, such as 5° C. to 45° C.

Immunoassays for detecting BoNT/A and/or BoNT/B are preferably either competitive or noncompetitive. Noncompetitive immunoassays are assays in which the amount of captured analyte (e.g., BoNT/A) is directly measured. In one preferred “sandwich” assay, for example, the capture agent (e.g., BoNT/A-neutralizing antibody) is bound directly or indirectly to a solid substrate where it is immobilized. These immobilized BoNT/A-neutralizing antibodies capture BoNT/A present in a test sample (e.g., a blood sample). The BoNT/A thus immobilized is then bound by a labeling agent, such as a BoNT/A-neutralizing antibody bearing a label. Alternatively, the second antibody may lack a label, but it may, in turn, be bound by a labeled third antibody specific to antibodies of the species from which the second antibody is derived. Free labeled antibody is washed away and the remaining bound labeled antibody is detected (e.g., using a gamma detector where the label is radioactive).

In another embodiment, a sandwich assay is produced that would simultaneously detect and distinguish between BoNT/A and BoNT/B. For example, the sandwich assay can be designed to measure the amount of remaining uncomplexed BoNT/A with respect to BoNT/B. In addition, the sandwich assay can be designed to detect both BoNT/A and BoNT/B. One of a pair of solid substrates could be bound to an antibody that binds only BoNT/A (e.g. 70A) and the other could be bound to an antibody that binds BoNT/B (e.g. 50B). The sample to be tested could be applied to the antibody-bound substrate. After washing, any toxin present in the sample that is bound to the 70A or 50B antibodies could be identified by a detection antibody that binds an epitope that is shared by BoNT/A and BoNT/B and which is different from either epitope bound by the BoNT/A-specific and BoNT/B-specific antibodies (e.g. 5A). For the purposes of this assay, the detection antibody could be affixed to a detectable group such as biotin, enzymes, nanoparticles, or other reporter technologies as described elsewhere herein.

In competitive assays, the amount of analyte (e.g., BoNT/A) present in the sample is measured indirectly by measuring the amount of an added (exogenous) analyte displaced (or competed away) from a capture agent (e.g., BoNT/A-neutralizing antibody) by the analyte present in the sample. In one competitive assay, a known amount of BoNT/A is added to a test sample with an unquantified amount of BoNT/A, and the sample is contacted with a capture agent, e.g., a BoNT/A-neutralizing antibody that specifically binds BoNT/A. The amount of added BoNT/A that binds to the BoNT/A-neutralizing antibody is inversely proportional to the concentration of BoNT/A present in the test sample.

The BoNT/A-neutralizing antibody can be immobilized on a solid substrate. The amount of BoNT/A bound to the BoNT/A-neutralizing antibody is determined either by measuring the amount of BoNT/A present in an BoNT/A-BoNT/A-neutralizing antibody complex, or alternatively by measuring the amount of remaining uncomplexed BoNT/A.

Other assays are encompassed in the present invention. For example, BoNT/A polypeptides or BoNT/A-neutralizing antibodies can also be detected and quantified by any of a number of other means well known to those of skill in the art. These include analytic biochemical methods such as spectrophotometry, radiography, electrophoresis, capillary electrophoresis, high performance liquid chromatography (IPLC), thin layer chromatography (TLC), hyperdiffusion chromatography, and the like. Immunological methods such as fluid or gel precipitin reactions, immunodiffusion (single or double), immunoelectrophoresis, radioimmunoassays (RIAs), enzyme-linked immunosorbent assays (ELISAs), immunofluorescent assays, and the like can be used to detect and quantify the antibodies of the present invention.

Western blot analysis and related methods can also be used to detect and quantify the presence of BoNT/A and/or BoNT/B polypeptides in a sample. The technique generally comprises separating sample products by gel electrophoresis on the basis of molecular weight, transferring the separated products to a suitable solid support, (such as a nitrocellulose filter, a nylon filter, or derivatized nylon filter), and incubating the sample with the antibodies of the present invention that specifically bind to BoNT/A and/or BoNT/B polypeptide. The antibodies specifically bind to the biological agent of interest on the solid support. These antibodies are directly labeled or alternatively are subsequently detected using labeled antibodies (e.g., labeled sheep anti-human antibodies where the antibody to a marker gene is a human antibody) which specifically bind to the antibody which binds BoNT/A and/or BoNT/B.

Labeling of BoNT/A and/or BoNT/B-Binding Antibodies.

The labeling agent can be, e.g., a monoclonal antibody, a polyclonal antibody, a protein or complex such as those described herein, or a polymer such as an affinity matrix, carbohydrate or lipid. Detection proceeds by any known method, including immunoblotting, western analysis, gel-mobility shift assays, tracking of radioactive or bioluminescent markers, nuclear magnetic resonance, electron paramagnetic resonance, stopped-flow spectroscopy, column chromatography, capillary electrophoresis, or other methods which track a molecule based upon an alteration in size and/or charge. The particular label or detectable group used in the assay is not a critical aspect of the invention. The detectable group can be any material having a detectable physical or chemical property. Such detectable labels have been well-developed in the field of immunoassays and, in general, any label useful in such methods can be applied to the present invention. Thus, a label is any composition detectable by spectroscopic, photochemical, biochemical, immunochemical, electrical, optical or chemical means. Useful labels in the present invention include magnetic beads (e.g. Dynabeads™), fluorescent dyes (e.g., fluorescein isothiocyanate, Texas red, rhodamine, and the like), radiolabels (e.g., ³H, ¹²⁵I, ³⁵S, or ³²P), enzymes (e.g., LacZ, CAT, horse radish peroxidase, alkaline phosphatase and others, commonly used as detectable enzymes, either as marker gene products or in an ELISA), and colorimetric labels such as colloidal gold or colored glass or plastic (e.g. polystyrene, polypropylene, latex, etc.) beads.

The label may be coupled directly or indirectly to the desired component of the assay according to methods well known in the art. As indicated above, a wide variety of labels may be used, with the choice of label depending on the sensitivity required, ease of conjugation of the compound, stability requirements, available instrumentation, and disposal provisions.

Non radioactive labels are often attached by indirect means. Generally, a ligand molecule (e.g., biotin) is covalently bound to the molecule. The ligand then binds to an anti-ligand (e.g., streptavidin) molecule which is either inherently detectable or covalently bound to a signal system, such as a detectable enzyme, a fluorescent compound, or a chemiluminescent compound. A number of ligands and anti-ligands can be used. Where a ligand has a natural anti-ligand, for example, biotin, thyroxine, and cortisol, it can be used in conjunction with the labeled, naturally occurring anti-ligands. Alternatively, any haptenic or antigenic compound can be used in combination with an antibody.

The molecules can also be conjugated directly to signal generating compounds, e.g., by conjugation with an enzyme or fluorophore. Enzymes of interest as labels will primarily be hydrolases, particularly phosphatases, esterases and glycosidases, or oxidoreductases, particularly peroxidases. Fluorescent compounds include fluorescein and its derivatives, rhodamine and its derivatives, dansyl, umbelliferone, etc. Chemiluminescent compounds include luciferin, and 2,3-dihydrophthalazinediones, e.g., luminol. For a review of various labeling or signal producing systems which may be used, see, U.S. Pat. No. 4,391,904, which is incorporated herein by reference.

Means of detecting labels are well known to those of skill in the art. Thus, for example, where the label is a radioactive label, means for detection include a scintillation counter or photographic film as in autoradiography. Where the label is a fluorescent label, it may be detected by exciting the fluorochrome with the appropriate wavelength of light and detecting the resulting fluorescence, e.g., by microscopy, visual inspection, via photographic film, by the use of electronic detectors such as charge coupled devices (CCDs) or photomultipliers and the like. Similarly, enzymatic labels may be detected by providing appropriate substrates for the enzyme and detecting the resulting reaction product. Finally, simple colorimetric labels may be detected simply by observing the color associated with the label. Thus, in various dipstick assays, conjugated gold often appears pink, while various conjugated beads appear the color of the bead.

Some assay formats do not require the use of labeled components. For instance, agglutination assays can be used to detect the presence of BoNT/A and/or BoNT/B peptides. In this case, antigen-coated particles are agglutinated by samples comprising the target antibodies. In this format, none of the components need be labeled and the presence of the target antibody is detected by simple visual inspection.

### Diagnosis/Treatment Kits

The antibodies of the invention are useful for the diagnosis, assessment and treatment of botulinum neurotoxin exposure. Moreover, the antibodies can be used to detect and or measure the amount of botulinum neurotoxin present in a biological sample using well-known methods such as, but not limited to, Western blotting and enzyme-linked immunosorbent assay (ELISA). Further, the antibodies can be used to immunoprecipitate and/or immuno-affinity purify their cognate antigen as described elsewhere herein.

Further included in the invention is a method of assessing the presence or degree of botulinum neurotoxin exposure in a mammal. This method is useful to diagnose botulinum neurotoxin exposure and is also useful for assessing the progress of the efficacy of treatment in an botulinum neurotoxin exposed mammal. The method comprises obtaining a biological sample from a mammal at risk of or having been exposed to a botulinum neurotoxin and assessing the amount of botulinum neurotoxin present in the biological sample, wherein a larger amount of botulinum neurotoxin, compared with the amount of botulinum neurotoxin from an otherwise identical mammal not at risk or exposed to a botulinum neurotoxin, is an indication that a botulinum neurotoxin is present in the mammal, and further wherein the presence of a botulinum neurotoxin in the mammal is related to the severity of the infected mammal. Such a method is useful in the diagnosis of botulinum neurotoxin exposure and in providing an assay for following up on the efficacy of botulinum neurotoxin treatments. That is, the amount of botulinum neurotoxin present in infected mammal can be evaluated before, during and after treatment and the efficacy of the treatment could thus be assessed. Further, such a method allows a determination of the presence and/or the severity of the disease in a mammal.

In another embodiment, this invention provides for kits for the treatment of botulism or for the detection/confirmation of a Clostridium botulinum infection. Kits will typically comprise one or more BoNT/A and/or BoNT/B-neutralizing antibodies of this invention. For diagnostic purposes, the antibody(s) can be labeled. In addition the kits will typically include instructional materials disclosing means of use BoNT/A and/or BoNT/B-neutralizing antibodies in the treatment of symptoms of botulism. The kits may also include additional components to facilitate the particular application for which the kit is designed. Thus, for example, where a kit contains a BoNT/A and/or BoNT/B-neutralizing-antibody wherein the antibody is labeled, the kit may additionally contain means of detecting the label (e.g. enzyme substrates for enzymatic labels, filter sets to detect fluorescent labels, appropriate secondary labels such as a sheep anti-human antibodies, or the like). The kits may additionally include buffers and other reagents routinely used for the practice of a particular method. Such kits and appropriate contents are well known to those of skill in the art.

Conveniently, the antibodies may be conjugated with markers, such as magnetic beads, which allow for direct separation, biotin, which can be removed with avidin or streptavidin bound to a support, fluorochromes, which can be used with a fluorescence activated cell sorter, or the like, to allow for ease of separation of the particular sample.

In accordance with the present invention, as described above or as discussed in the Examples below, there can be employed conventional clinical, chemical, cellular, histochemical, biochemical, molecular biology, microbiology and recombinant DNA techniques which are known to those of skill in the art. Such techniques are explained fully in the literature.

The invention should not be construed to be limited solely to the assays and methods described herein, but should be construed to include other methods and assays as well. One of skill in the art will know that other assays and methods are available to perform the procedures described herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### Example 1

### Generation of the 5A Hybridoma

The native human antibody repertoire holds unexplored potential for the development of novel monoclonal antibody therapeutics. Production of a monoclonal antibody includes the fusion of an immortal cell with a primary B-lymphocyte to generate a hybridoma that secretes a monoclonal antibody. A useful cell line for generating a human monoclonal antibody is the SP2/mL-6 MPT2 cell line, which is a murine cell line that ectopically expresses murine interleukin-6 (mIL-6) and human telomerase (hTERT). It has been demonstrated that such a cell line efficiently forms stable human antibody-secreting hybridomas through cell fusion with primary human B-lymphocytes. The hybrid cells are able to maintain secretion of human antibodies derived from the primary B-lymphocytes through multiple rounds of cloning.

The cells were cultured using standard methods in the art. For example, confluent cells are split 1:10 into fresh medium Gibco Advanced RPMI with 1% IFS, with L-glutamine, penicillin and streptomycin, and grown for 4-5 days. Supernatants were harvested by centrifugation of the cell suspension and then filtered through a 22 micron syringe filter. Supernatants were then concentrated prior to testing in mice 10-20 fold using an Amicon (Amersham) concentrator. Concentrated supernatants were tested for BoNT-specific antibody by ELISA prior to testing in mice.

Botulinum toxin ELISAs were performed following standard techniques. Briefly, purified BoNT/A toxin was used, or recombinant, E.-coli-expressed 50-KD C-terminal toxin domains (HC50) from BoNT/A or BoNT/B. 5 micrograms/ml BoNT/A toxin or 5 micrograms/ml HC50 domain peptides were applied to 96-well ELISA plates, 100 microliters/well, and incubated overnight at 4° C. Plates were blocked with PBS/5% calf serum/3% goat serum (Sigma, St. Louis, Mo.) for 1 hour at 37° C. Washes were performed with PBS/Tween 20 0.5%. Undiluted hybridoma supernatants were added to the wells for testing. After washing, an HRP-conjugated antibody selected from the following list was used for detection: Goat polyclonal anti-human IgM, (Sigma), mouse monoclonal anti-human IgG (clone JDC-10) (Southern Biotech, Birmingham Ala.) or a goat polyclonal anti-human IgG (Southern Biotech). Detection was performed with OPD substrate (Sigma).

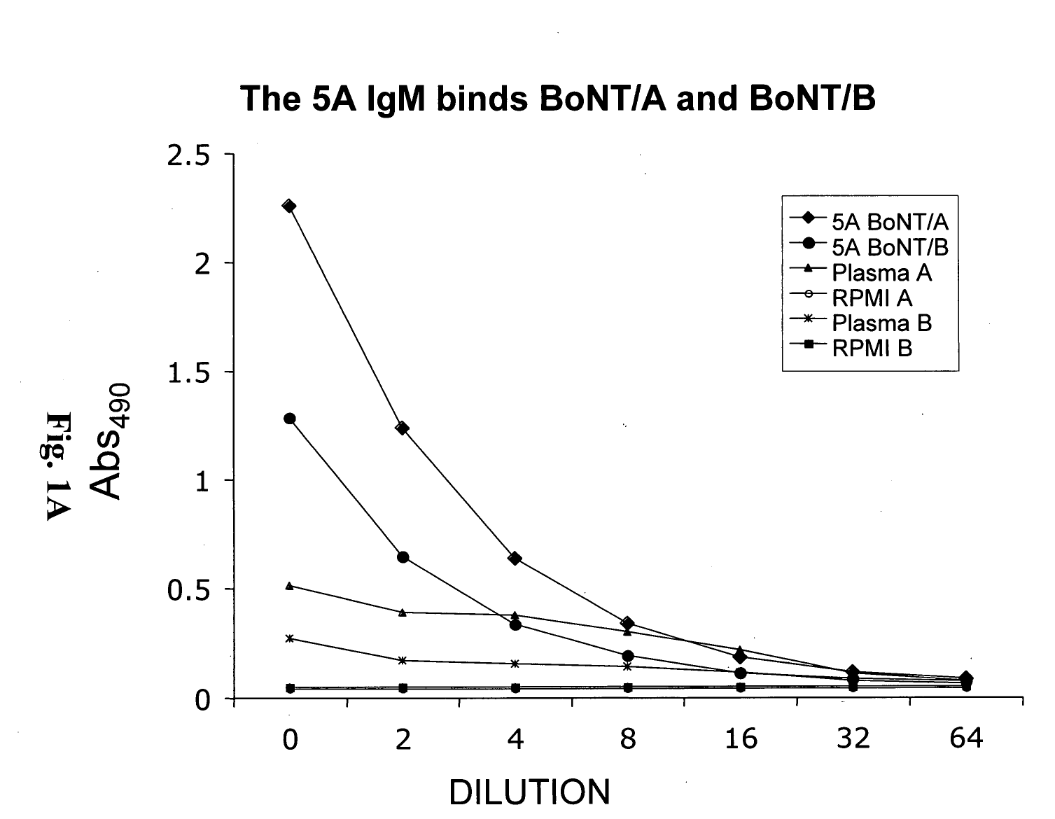

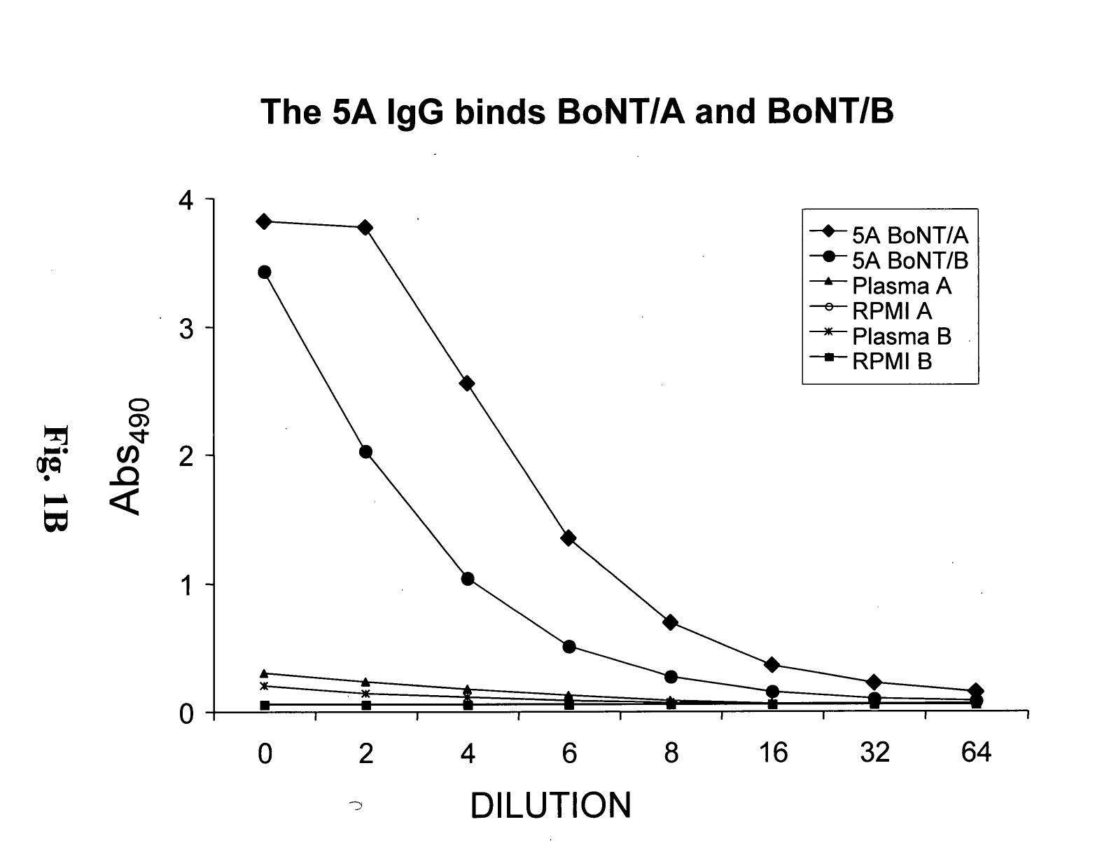

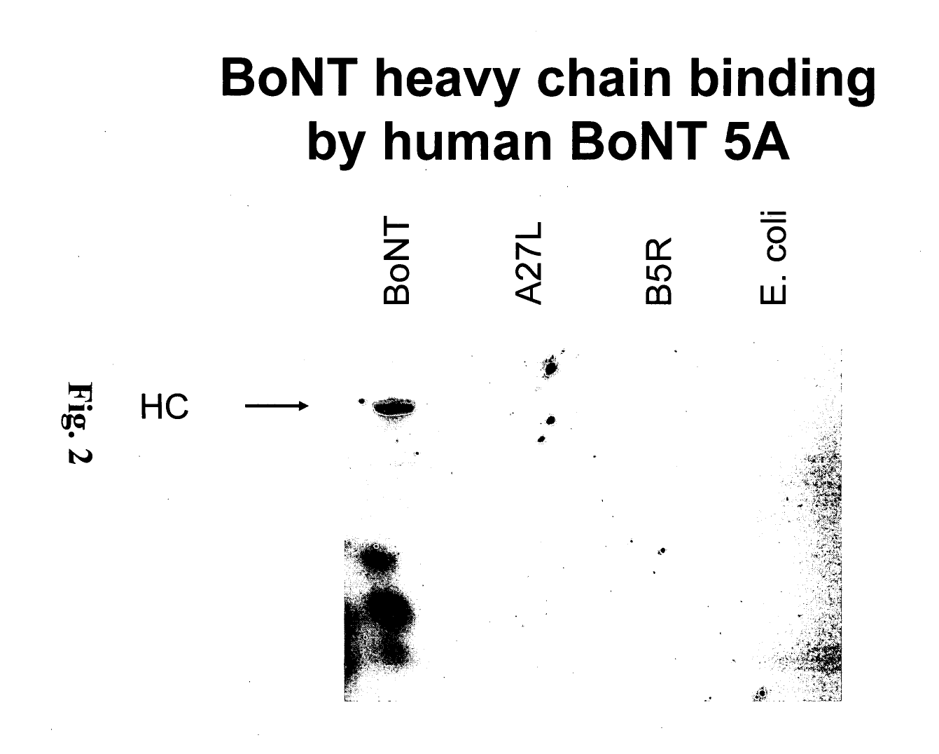

The 5A hybridoma was generated as follows. Peripheral blood lymphocytes from a volunteer donor were stimulated with pokeweed mitogen (5 micrograms/ml) and purified BoNT/A (10 micrograms/ml) for 5 days. The peripheral blood lymphocytes were then fused to the SP2/mL-6 MPT2 cell line and selected with HAT using standard techniques. Hybrid cell pools were tested for human IgM antibodies secreted into their supernatants that specifically bound BoNT/A by ELISA. Cells in a positive pool were cloned by limiting dilution. The initial, first-round clone was identified as an IgM kappa antibody. The specificity of the antibody for BoNT/A was verified by a limiting dilution ELISA. It was also observed that the 5A antibody was specific for the BoNT/B as measured by was determined by limiting dilution ELISA (FIG. 1). The specificity for the BoNT/A heavy chain was identified using Western Blotting analysis (FIG. 2). A representative third-round clone was identified and named 5-3-F11-2-F8-C2.

### Example 2

### Generation of the 70A Hybridoma

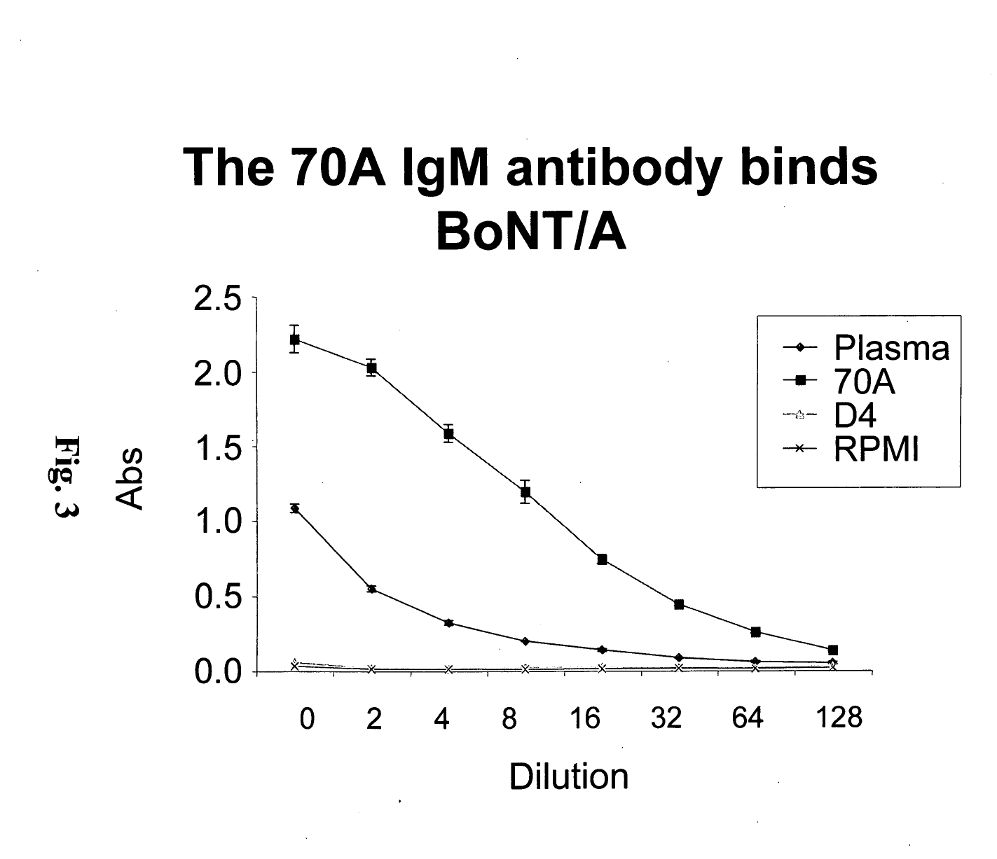

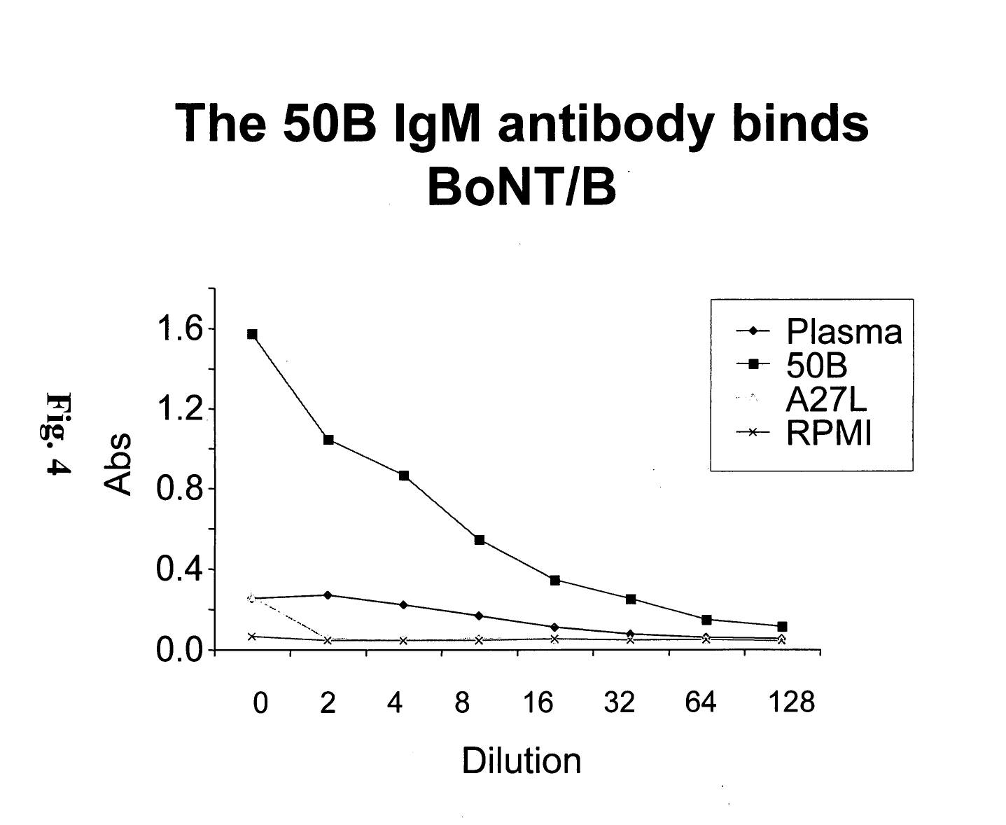

The 70A hybridoma was generated as follows. Peripheral blood lymphocytes from a volunteer donor were stimulated on a CD40 ligand-expressing cell monolayer (tCD40L (Schultze et al., 1997)) with IL-4 (2 ng/ml) for 14 days. The peripheral blood lymphocytes were then fused to the SP2/mL-6 MPT2 cell line and selected with HAT using standard techniques. Hybrid cell pools were tested for human IgM antibodies secreted into their supernatants that specifically bound BoNT/A by ELISA. Cells in a positive pool were cloned by limiting dilution. A first-round clone was identified as an IgM that specifically bound to BoNT/A (FIG. 3). A representative third-round clone was identified and named 70A-D5.

### Example 3

### Generation of the 50B Hybridoma

The 50B hybridoma was generated as follows. Peripheral blood lymphocytes from a volunteer donor were stimulated on a CD40Ligand-expressing cell monolayer (tCD40L (Schultze et al., 1997)) with IL-4 (2 ng/ml) for 14 days, then fused to the (c-clone hTERT cell line, a version of SP2/mL-6 MPT2 cell line that was selected for high mIL-6 expression) and selected with HAT using standard techniques. Hybrid cell pools were tested for human IgM antibodies secreted into their supernatants that specifically bound BoNT/B by ELISA. Cells in a positive pool were cloned by limiting dilution. A first-round clone specifically binding BoNT/B was identified as by ELISA (FIG. 4). A representative third-round clone was identified and named 50B-B8.

### Example 4

### Neutralization Activity of the Antibodies

The following results demonstrated the neutralization activity of the antibodies. Briefly, mouse protection assays were performed with 25 gram Swiss-Webster mice. The specified amount of BoNT/A or BoNT/B was mixed for 1 hour at room temperature with concentrated hybridoma supernatant (IgM) prior to tail-vein injection. For testing the antibody in mice, cell supernatants were concentrated 10-20 fold using an Amicon concentrator (Millipore, Billerica, Mass.) prior to testing in mice. The concentrated protein was quantitated using a NanoDrop spectrophotometer (NanoDrop Technologies, Wilmington, Del.).

It was observed that the 70A antibody neutralized BoNT/A in vivo, thus protecting a mouse from an otherwise lethal dose of toxin. In this experiment, 4 Swiss-Webster mice that received 10 pg BoNT/A with a control cell culture medium were dead within 48 hrs. It was observed that mice (n=6) that received the 10 pg BoNT/A mixed with 100 microliters of concentrated hybridoma supernatant containing approximately 30 micrograms of the 70A antibody survived. In contrast, mice that received BoNT/A with either a control cell medium supernatant (n=6) or concentrated cell culture supernatant containing 5A antibody (n=6) died with 1-2 days. Similarly, the 50B antibody did not protect mice from a lethal dose of BoNT/B.

### Example 5

### Cloning of Antibodies

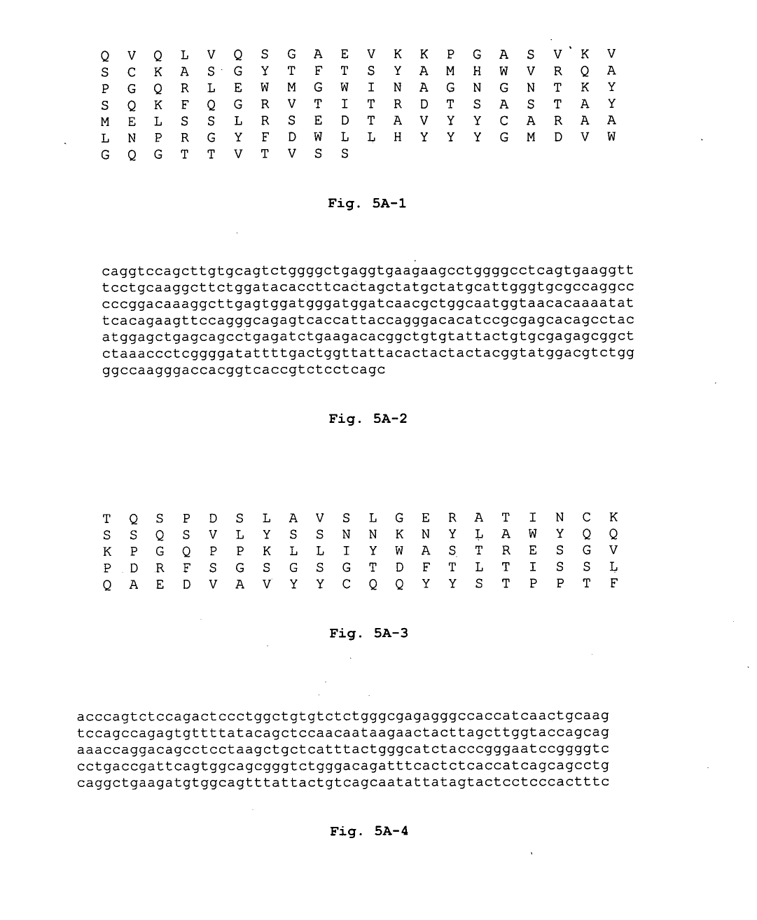

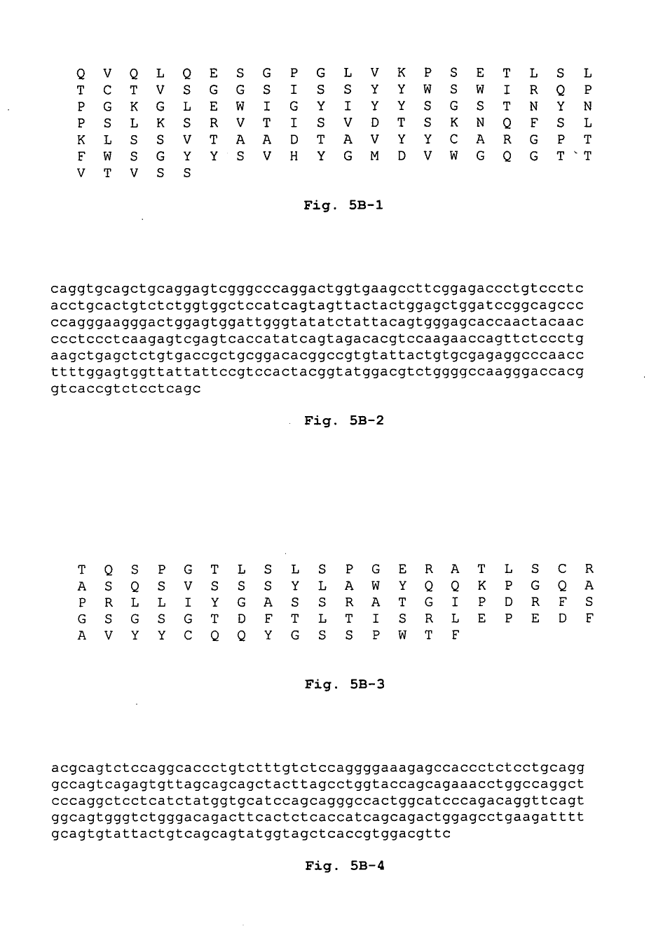

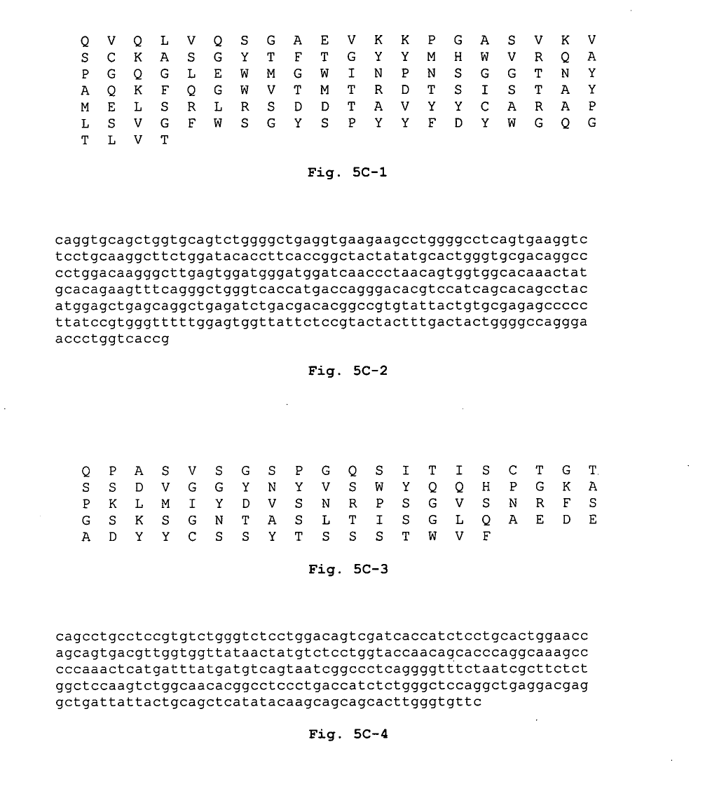

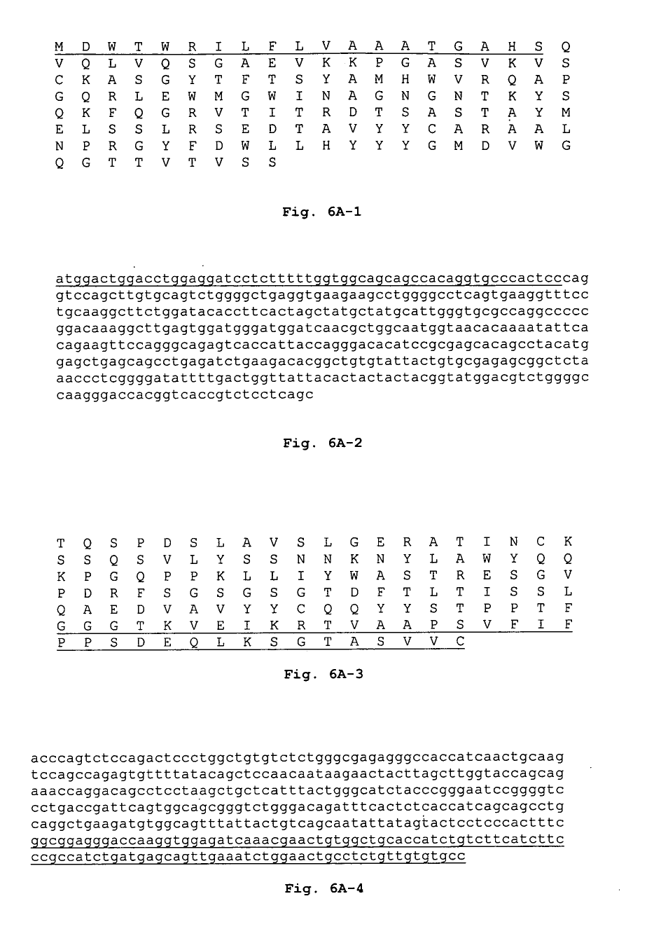

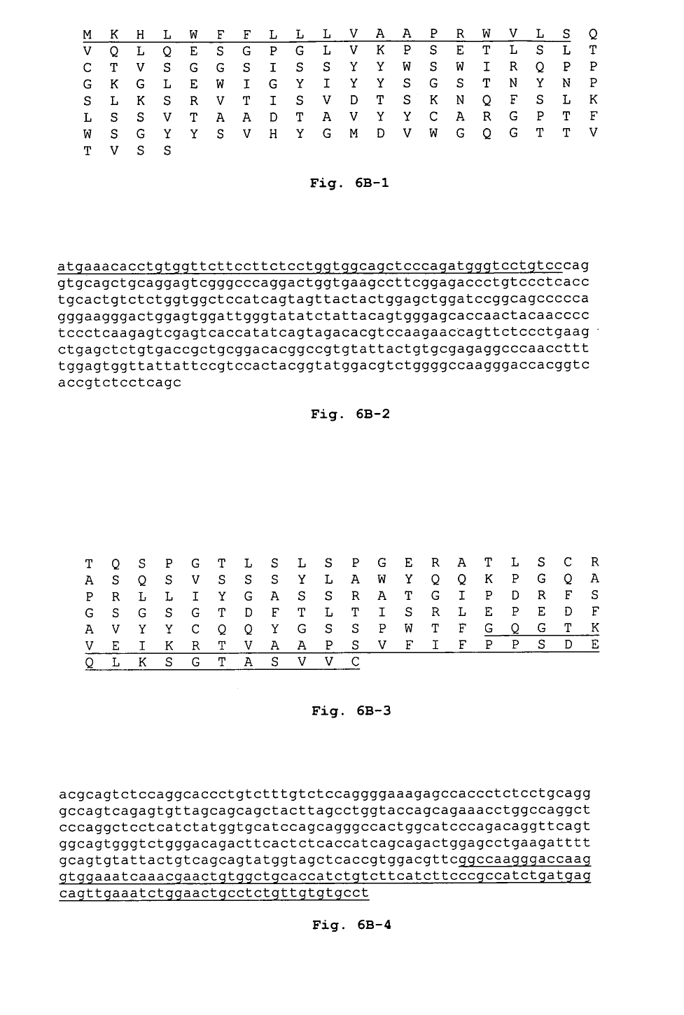

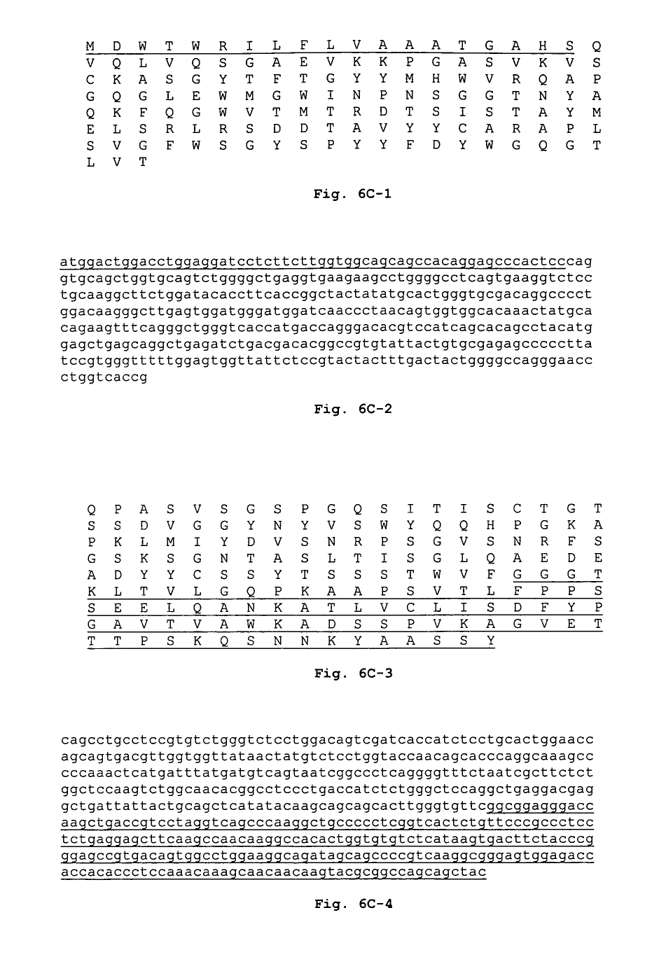

cDNAs of the heavy chain and light chain variable domains of the 5A, 70A and 50B antibodies were cloned from the hybridomas described elsewhere herein with standard RT-PCR techniques (FIGS. 5 and 6). Oligonucleotides for the heavy chain N-terminal regions, including the variable domain, were taken from Campbell et al. (1992, Mol. Immunol. 29:193-203). Oligonucleotides for the kappa light chain regions were taken from Marks et al. (1991, Eur J Immunol. 21:985-91). Oligonucleotides for the lambda light chain were taken from Coronella et al. (2000 Nucleic Acids Res. 28:85). Following RT-PCR, the amplified DNA fragments were cloned into the plasmid pCRTOPO 2.1 (Invitrogen, Carlsbad, Calif.) and sequenced.

The 5A heavy chain variable domain was amplified with the primers: XK-VH1ldr CACTCGAGCCGCCATGGACTGGACCTGGA (SEQ ID NO:1) and Cmu-R4 CCTGAGGAGACGGTGACC (SEQ ID NO:2).

The 5A kappa light chain variable domain was amplified with the primers VK4F GACATCCAGCTGACCCAGTCTCC (SEQ ID NO:3) and Kappa768 RSX GTCGACCTCGAGGTCAGGCTGGAACTGAGGAG (SEQ ID NO:4).

The 70A heavy chain variable domain was amplified with the primers: XK-VH1ldr CACTCGAGCCGCCATGGACTGGACCTGGA (SEQ ID NO:5) and Cmu-R4 CCTGAGGAGACGGTGACC (SEQ ID NO:6).

The 70A lambda light chain variable region was amplified with the primers VL7/8B CCGCAGDCTGTGGTGACYCAGGAGCC (SEQ ID NO:7) and VL9B CCGCAGCCWGKGCTGACTCAGCCMMCC (SEQ ID NO:8) and reverse primers CL2R CGCCGTCTAGAACTATGAACATTCTGTAG (SEQ ID NO:9) (for RT reaction) and LnestR GCACTAATGCGTGACCTGGCAGCTGT (SEQ ID NO:10) (for PCR). The primer sequences represented in SEQ ID NO:7 and SEQ ID NO:8 are degenerate primers, which at some designated positions in the primer sequence can contain different bases, thereby creating a population of oligos that are heterogeneous with respect to the designated positions. As such, D=A, G or T; Y═C or T, W=A or T; K=G or T; M=A or C at the degenerate positions.

The 50B heavy chain variable domain was amplified with the primers: XK-VH4ldr CACTCGAGCCGCCATGGACTGGACCTGGA (SEQ ID NO:11) and Cmu-R4 CCTGAGGAGACGGTGACC (SEQ ID NO:12).

The 50B kappa light chain variable domain was amplified with the primers VK3F GAAATTCAGCTGACGCAGTCTCC (SEQ ID NO:13) and Kappa R3 GTTATTCAGCAGGCACACAACAG (SEQ ID NO:14).

Conversion of the 5A IgM antibody to an IgG₁ antibody of the same specificity was accomplished by standard molecular methods. The coding sequence of the heavy chain variable region was amplified by PCR using the primers BotH1 FNEKozak GCTACGAATTCGCCGCCATGGACTGGACC (SEQ ID NO:15) and BotH1H3R2 TATGAAGCTTGTCGAGGAGACGGTGACCGTG (SEQ ID NO:16) with the pCR2.1 TOPO 5A HC plasmid. This PCR introduced a HindIII site at the variable region/constant region boundary of the HC; this fragment was then subcloned into a plasmid pBSK1 IgG1, which contains a HindIII-XhoI DNA sequence encoding the IgG1 constant domain region, taken from phc-huCg1 (McLean et al., 2000 Mol Immunol. 37:837-45), creating a full-length IgG1 cDNA. This cDNA was cloned into the retroviral transfer vector pBabe Puro (Morgenstern and Land, 1990 Nucleic Acids Res. 18:3587-96).

A full-length 5A kappa cDNA was obtained by performing RT-PCR with the primers BotK sigF-C GGATCCAGATCTGCCGCCACCATGGACATGAGAGTCCTCGCTC (SEQ ID NO:17), cloning the PCR product into pCR2.1 TOPO, and subcloning the insert into the retroviral transfer vector pBabe neo (Morgenstern and Land, 1990 Nucleic Acids Res. 18:3587-96). The plasmids pBabe Puro 5A HC, pBabe Neo 5A LC, and pCL Ampho were co-transfected into 293T cells by standard techniques using Fugene 6 (Roche, Indianapolis, Ind.). Retroviral supernatants were applied to CHO cells that were selected with puromycin (4 micrograms/ml) and G418 (500 micrograms/ml). High-expressing, stable clones were obtained by single cell cloning in 96 well plates and identified by ELISA.

The disclosures of each and every patent, patent application, and publication cited herein are hereby incorporated herein by reference in their entirety.

While the invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. An isolated antibody comprising: 1) an antibody heavy chain variable domain having an amino acid sequence of SEQ ID NO:26; and 2) an antibody light chain variable domain having an amino acid sequence of SEQ ID NO:28.

2. (canceled)

3. An isolated antibody that specifically binds to an epitope specifically bound by an antibody comprising: 1) an antibody heavy chain variable domain having an amino acid sequence of SEQ ID NO:22; and 2) an antibody light chain variable domain having an amino acid sequence of SEQ ID NO:24.

4. An isolated antibody comprising: 1) an antibody heavy chain variable domain having an amino acid sequence having at least 80% homology to SEQ ID NO:22; and 2) an antibody light chain variable domain having an amino acid sequence having at least 80% homology to SEQ ID NO:24.

5. An isolated antibody that specifically binds to an epitope specifically bound by an antibody comprising: 1) an antibody heavy chain variable domain having an amino acid sequence of SEQ ID NO:18; and 2) an antibody light chain variable domain having an amino acid sequence of SEQ ID NO:20.

6. An isolated antibody comprising: 1) an antibody heavy chain variable domain having an amino acid sequence having at least 80% homology to SEQ ID NO:18; and 2) an antibody light chain variable domain having an amino acid sequence having at least 80% homology to SEQ ID NO:20.

7. The antibody of claim 1, wherein said antibody is a single chain Fv (scFv), a Fab, a (Fab′)₂ or a (scFv′)₂.

8. An isolated polypeptide, wherein said polypeptide comprises an amino acid sequence selected from the group consisting of:
a) SEQ ID NO:18 or an amino acid sequence having at least 80% homology to SEQ ID NO: 18;
b) SEQ ID NO:20 or an amino acid sequence having at least 80% homology to SEQ ID NO:20;
c) SEQ ID NO:22 or an amino acid sequence having at least 80% homology to SEQ ID NO:22;
d) SEQ ID NO:24 or an amino acid sequence having at least 80% homology to SEQ ID NO: 24;
e) SEQ ID NO:26 or an amino acid sequence having at least 80% homology to SEQ ID NO: 26; and
f) SEQ ID NO:28 or an amino acid sequence having at least 80% homology to SEQ ID NO: 28.

9. An isolated nucleic acid molecule, wherein said nucleic acid molecule comprises a sequence selected from the group consisting of:
a) SEQ ID NO:19 or a nucleotide sequence having at least 80% homology to SEQ ID NO: 19;
b) SEQ ID NO:21 or a nucleotide sequence having at least 80% homology to SEQ ID NO:21;
c) SEQ ID NO:23 or a nucleotide sequence having at least 80% homology to SEQ ID NO:23;
d) SEQ ID NO:25 or a nucleotide sequence having at least 80% homology to SEQ ID NO: 25;
e) SEQ ID NO:27 or a nucleotide sequence having at least 80% homology to SEQ ID NO: 27; and
f) SEQ ID NO:29 or a nucleotide sequence having at least 80% homology to SEQ ID NO: 29.

10. A method of neutralizing BoNT/A in a subject in need thereof, said method comprising administering to said subject a therapeutically effective amount of an antibody according to claim 1.

11. A method of neutralizing BoNT/A in a subject in need thereof, said method comprising administering to said subject a therapeutically effective amount of an antibody according to claim 2.

12. The method of claim 10 or 11, wherein said antibody wherein said antibody is a single chain Fv (scFv), a Fab, a (Fab′)₂ or a (scFv′)₂.

13. A method of detecting and distinguishing between BoNT/A and BoNT/B, the method comprising: binding a first antibody that specifically binds to either BoNT/A or BoNT/B to a solid phase surface; applying a test sample over said first antibody; washing away unbound material from said first antibody; applying a second antibody that specifically binds an epitope that is shared by BoNT/A and BoNT/B.

14. The method of claim 13, wherein said first antibody specifically binds to BoNT/A, further wherein said first antibody comprises: 1) an antibody heavy chain variable domain having an amino acid sequence of SEQ ID NO:26; and 2) an antibody light chain variable domain having an amino acid sequence of SEQ ID NO:28

15. The method of claim 13, wherein said first antibody specifically binds to BoNT/B, further wherein said first antibody comprises: 1) an antibody heavy chain variable domain having an amino acid sequence of SEQ ID NO:22; and 2) an antibody light chain variable domain having an amino acid sequence of SEQ ID NO:24.

16. The method of claim 13, wherein said second antibody binds to an epitope that is shared by BoNT/A and BoNT/B, further wherein said second antibody comprises: 1) an antibody heavy chain variable domain having an amino acid sequence of SEQ ID NO:18; and 2) an antibody light chain variable domain having an amino acid sequence of SEQ ID NO:20.
